(19) United States
(12) Patent Application Publication
(10) Pub. No.: US 2024/0299431 A1
(43) Pub. Date: Sep. 12, 2024

(54) NEW SYNTHETIC AGONISTS OF TLR4 RECEPTOR

(71) Applicant: UNIVERSITÀ DEGLI STUDI DI MILANO - BICOCCA, Milano (IT)

(72) Inventors: Francesco PERI, Milano (IT); Alessio ROMERO, Milano (IT); Simona D'AMATO, Milano (IT)

(21) Appl. No.: 18/575,462

(22) PCT Filed: Jul. 19, 2022
(86) PCT No.: PCT/IB2022/056615

§ 371 Date: Dec. 29, 2023

(30) Foreign Application Priority Data
Jul. 22, 2021 (IT) 102021000019544
Mar. 29, 2022 (IT) 102022000006149

(51) Int. Cl. A61K 31/7024; A61K 39/39; A61K 45/06; A61P 37/06; C07H 1/02; C07H 23/00

(52) U.S. Cl. CPC A61K 31/7024; A61K 39/39; A61K 45/06; A61P 37/06; C07H 1/02; C07H 23/00

(57) ABSTRACT

The present invention relates to new synthetic molecules with agonist activity of human Toll-like Receptor 4 (TLR4), compositions comprising them and uses thereof for the treatment of diseases in which it is useful to induce or increase an immune response. These new syntethic molecules differ from other similar agonists due to the simplicity of the formula, the ease and cheapness of preparation and the possibility of further chemical processing to modify the physicochemical properties and allow conjugation to other molecules (for example protein antigens).

The present invention relates to new synthetic molecules with agonist activity of human Toll-like Receptor 4 (TLR4), compositions comprising them and uses thereof, in particular for the treatment of diseases in which it is useful to induce or increase an immune response.

### PRIOR ART

Innate immunity is the first line of defense of higher organisms against pathogens and cell damage. It is based on the recognition of specific molecular structures associated with pathogens or cell damage (respectively PAMPs and DAMPs) by specific protein receptors. Such receptors are known as pattern recognition receptors (PRRs) and may be of various typologies, depending on their localization in-cell, in cytosol or on the membrane, and on their function.

Among the most studied receptors are those of the Toll-Like Receptor (TLR) family, whose main role is to recognize different PAMPs, alerting the body to the presence of pathogens through inflammation, recruiting other immune cells to fight infection and thus initiating the process of developing adaptive immunity, which is the most appropriate and specific defense for such menaces.

In fact, innate immunity response to pathogens can be decisive in determining both the nature and the intensity of adaptive immunity response.

For this reason, the development of TLR activators (agonists) has pharmaceutical relevance where an inflammatory stimulus is beneficial from a therapeutic point of view: examples are drugs for cancer immunotherapy and vaccine adjuvants. In the first case, pro-inflammatory activity can lead to the reactivation of the immune system in the tumor environment, which can therefore destroy the tumor (Bhatia S, Miller N J, Lu H, et al.

Intratumoral G100, a TLR4 agonist, induces antitumor immune responses and tumor regression in patients with Merkel cell carcinoma. Clin Cancer Res. 2019; 25(4):1185-1195. doi:10.1158/1078-0432.CCR-18-0469).

In the second case, a pro-inflammatory activity is advantageous because modern vaccines no longer use whole inactivated pathogens, but subunits thereof, which are unable to stimulate a correct inflammatory response without adjuvation. To date, there are various small molecules able to bind and activate TLR receptors, and some of those are used as adjuvants such as: imidazoquinoline TLR7/8 agonists, like imiquimod and resiquimod, as well as Pam2CS-type TLR2/TLR6 agonist and TLR4 agonists such as monophosphoryl lipid A (MPL) and aminoalkyl glucosaminide-4-phosphates (AGPs, also referred to as Corixa compounds, CRX).

Among TLRs, TLR4 is of high pharmacological interest: its activation is the most efficient way to stimulate innate and adaptive immunity. Indeed, TLR4 exhibits two different and distinct cellular mechanisms of functioning that lead to the release of a larger and more heterogeneous set of proinflammatory cytokines, causing a more complete immune response.

The natural agonist of TLR4 is lipopolysaccharide (LPS), the main component of the outer membrane of Gram-negative bacteria. It can be divided into three parts: a long polysaccharide chain, called O-Antigen, a shorter oligosaccharide called Core and finally Lipid A (lpd A), the immunogenic portion of the molecule, formed by two glucosamines to which are commonly linked two phosphates and a variable number of acyl chains.

Lipid A agonistic activity is based on its binding affinity (ability to bind) to the TLR4 co-receptor, Myeloid Differentiation factor 2, MD-2, with the entailed formation of the (TLR4/MD-2/LPS)₂ complex on the surface of innate immunity cells, i.e. macrophages and dendritic cells.

The activation process of the TLR4 receptor by LPS begins with the interaction of individual LPS molecules or aggregates in solution with Lipid Binding Protein (LBP), forming a complex with an LPS molecule. Thereafter, the LPS molecule is transferred from LBP to co-receptor CD14, which in turn transfers it from MD-2.

However, Ipd A is toxic even in quantities of the order of picograms, and consequently it cannot be used pharmacologically (Molinaro A, Holst O, Lorenzo F Di, et al. Chemistry of lipid a: At the heart of innate immunity. Chem—A Eur J. 2015; 21(2):500-519. doi:10.1002/chem.201403923).

Synthetic and natural molecules with a structure similar to lipid A, but with attenuated endotoxicity, are interesting candidates as vaccine adjuvants in the perspective of maintaining immunostimulatory activity while eliminating the toxic effects.

Monophosphoryl lipid A (MPL) is a molecule identical to lipid A, except for the absence of the C₁ phosphate group. Despite being so similar, the inflammatory activity is only 0.1% of that of the natural molecule, and its pharmacological profile is so good that it has been approved by the FDA for use as a vaccine adjuvant. The molecule is presently used in Cervarix and Fendrix vaccines. However, the MPL adjuvant used nowadays is chemically heterogeneous, as produced directly from natural LPS.

In addition, the synthesis of these disaccharide compounds is very long and complex, resulting in a final price of about 200 eur/mg. For this reason, it is interesting to develop further lipid A analogs with a monosaccharide structure, sharing the same advantageous features of the known analogs or being even improved, preferably obtainable through a simpler synthetic route.

An example of monosaccharidic lipid A analogs known in the art is represented by the synthetic compounds named AGPs (also known as CRX adjuvants, Corixa) comprise a monosaccharide unit linked by glycosidation to a unit of an aminoalkyl aglycone N-acylate.

AGPs are potent agonists of TLR4 and are chemically homogeneous being produced by chemical synthesis.

Further, simpler, analogs of Lipid A that are still effective in the activation of TLR4 comprise monophosphorylated monosaccharide derivatives mimicking the reducing portion or the non-reducing portion of Lipid A (scheme below).

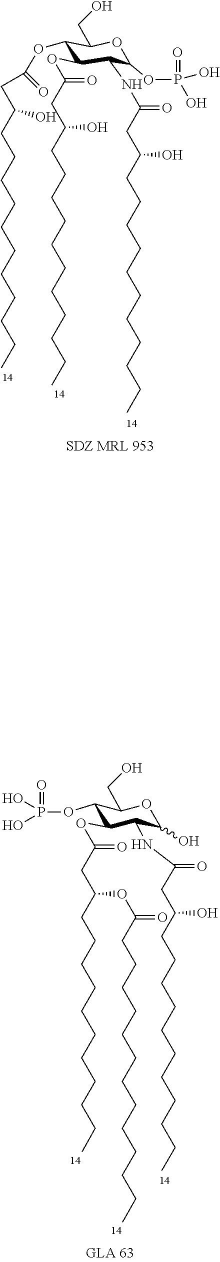

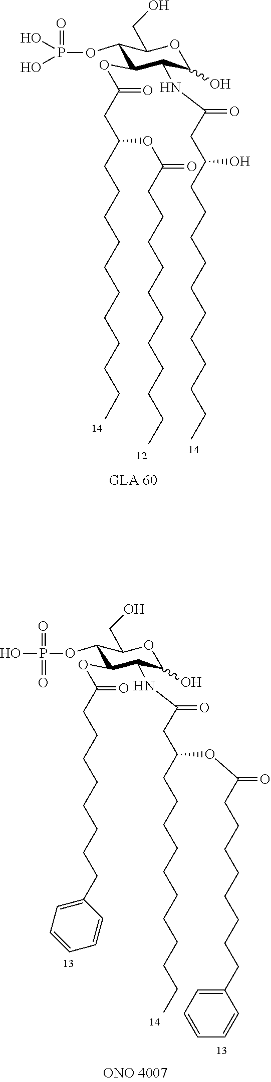

An other example of compounds known in the art is represented by compounds GLA 63 and GLA60 (scheme above) comprise a glucopyranoside skeleton, phosphorylated in position C₄, a 14-Carbon linear chain in C₂ and a branched chain in C₃ (14+14 in GLA 63 or 14+12 Carbons in GLA 60) (Motohiro Matsuura, Makoto Kiso and Akira Hasegawa Infect. Immun. 1999, 67(12), 6286-6292). These monosaccharides that partially mimic lipid A and mimic the monosaccharide lipid X, which is a biosynthetic precursor of lipid A, are active in stimulating TLR4-dependent production of cytokines TNF-α and IL-6, in both murine and human cells.

Also compound SDZ MRL 953, known in the art, showed a potent activity in stimulating the release of inflammatory cytokines such as interleukin-6 (IL-6), interleukin-8 (IL-8) and TNF-α factor in murine macrophages and neutrophil granulocytes, concomitantly exhibiting a toxicity reduced of a factor of at least 104 in galactosamine-sensitized mice compared to the parent endotoxin (Salmonella abortus equi).

In experimental microbial infection models, the compound proved to have a highly protective effect when administered prophylactically either once or thrice in myelo-suppressed or immunocompetent mice.

Doses effective to reach 50% of response with SDZ MRL 953 vary depending on the infective agent and administration route. In all cases, however, EC₅₀ obtained are about 103 times greater than those obtained with endotoxin Salmonella abortus equi.

However, thanks to the low toxicity, the therapeutic indexes of this molecule, expressed, e.g., as LD₂₅/ED₇₅ were significantly improved compared to the endotoxin and range from about 5 to >500, depending on the infective agent and the administration route.

The compound also proved efficient in inducing tolerance to endotoxins: repeated dosages of the compound induce a transient resistance (>1 week) to endotoxin-related lethal risks.

All these positive results were also confirmed in a model of advanced sepsis caused by Escherichia coli, in which antibiotic therapy had already proved inefficient: pre-treatment with one dose of SDZ MRL 953 one day prior to microbial inoculation dramatically increased the curative effects of antibiotics administered. For this reason, long-term survival was significantly increased with incremental doses of the immunostimulant in combined therapy.

Due to the tolerability demonstrated by SDZ MRL 953 in animals and in vitro, this compound was subsequently tested in human models.

On the basis of the known anti-tumour activity of Salmonella abortus equi endotoxin linked to its immunostimulating properties, Kiani et al. (A. Kiani, A. Tschiersch, E. Gaboriau, F. Otto, A. Seiz, H.-P. Knopf, P. Stutz, L. Farber, U. Haus, C. Galanos, R. Mertelsmann, and R. Engelhardt, Blood, 1997, 1673-1683) conducted a randomized double-blind phase I trial with control medium, administering SDZ MRL 953 in tumour-affected patients in order to assess firstly its biological effects and its safety of administration in humans and, secondly, its influence on the reaction to a subsequent endotoxin (LPS) addition.

SDZ MRL 953 administration proved safe and of excellent tolerability. The same SDZ MRL 953 increases granulocyte counts and serum levels of G-CSF and interleukin-6 (IL-6), but not of pro-inflammatory cytokines TNF-α, IL-1b, and IL-8.

Therefore, SDZ MRL 953 has three relevant features, i.e., 1) a high tolerability and low toxicity, 2) the ability to induce G-CSF production, and, as a result, 3) the ability to stimulate an aspecific immune resistance expressed by an increased group of primary defenses in cells.

In spite of these positive results encouraging in the clinical use of SDZ MRL 953, the mechanism of action of this molecule has not yet been studied in molecular detail.

The synthesis of SDZ MRL 953 is complex due to the fact that the glucosamine core binds, in positions C₂, C₃ and C₄, chains of 3(R)-hydroxymyristic acid as pure enantiomer. 3-hydroxymyristic acid is commercially available as racemate, whereas the pure enantiomer 3(R)-hydroxymyristic acid needs to be isolated from the racemate prior to use in the synthesis of SDZ MRL 953.

WO2019/092572 discloses a further class of compounds, with a triacylated monophosphoryl glucosamine core and one phosphate group on position C₁ in particular FP112.

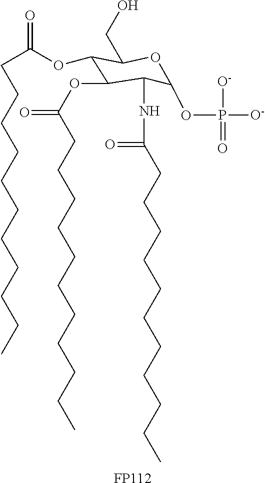 [SMILES: "FP112" ; CCCCCCCCCCCC(=O)NC1C(OP(=O)([O-])[O-])OC(CO)C(OC(=O)CCCCCCCCCCC)C1OC(=O)CCCCCCCCCCC]

FP112 is a compound similar to the reducing end of Ipd A and to SDZ MRL 953, but with three totally saturated and unsubstituted acyl chains. This compound is significantly easier to synthesize than the ones known in the art as the production of optically pure acyl chains is not required for its synthesis.

FP112 has an excellent pharmacological profile, as it is able to stimulate the release of numerous proinflammatory cytokines, the most notable of which are IL-1α, IL-1β, IL-6, TNF-α and IFNβ.

FP112 has been extensively tested in vitro on Hek-Blue, Raw-Blue, THP-1 cells, always demonstrating low toxicity and good pro-inflammatory activity already at a concentration of 10 μM.

### SUMMARY OF THE INVENTION

The authors of the present invention have identified a group of new compounds with a triacylated monophosphoryl glucosamine core, different from the ones known in the art, that are effective agonists of the TLR4 receptor. Advantageously, said new compounds are more versatile than the ones described in the art as they can be functionalized with various groups of interest. The authors of the present invention have also developed a new method of synthesis of said new compounds as well as of other compounds known in the art, that is simpler, faster and less expensive than the methods disclosed in the art.

The Authors of the present invention herein provide new compounds of formula 1

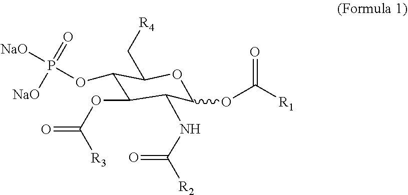

wherein R₁ is a saturated C₅-C₁₅ alkyl chain,

wherein R₂ is a saturated C₅-C₁₅ alkyl chain,

wherein R₃ is a saturated C₅-C₁₅ alkyl chain,

wherein R₄ is any substituent known by the skilled person that can be linked by means of a bond between C₆ and a suitable atom and/or any substituent which possesses an oxygen or a nitrogen atom that can bind to C₆.

said compounds are apparently similar to the compounds disclosed in WO2019/092572, but have several advantages over the same. A first advantage is represented by the fact that the molecular structure of the compounds of formula 1 makes them more stable: in fact, the phosphate group, which is known to be one of the best leaving groups in organic chemistry, in position C₄ in the compounds of formula 1 has proven to be much more stable than the phosphate group in C₁, present in the compounds of the prior art discussed above.

As an example, compounds of formula 1 are more stable in the desilylation reaction (reaction 5 of FP20 synthesis and reaction 6 of the FP11 synthesis).

During the synthesis of FP112 the lability of C₁ phosphate was a major issue and phosphate degradation was observed also operating at mild conditions, with the formation of two major dephosphorylated byproducts (showed in the experimental section). Moreover, the purification yield id lower in the case of FP11, averaging toward 50%.

In FP20 the desilylation proceded with good yields, without the problem of anomeric phosphate cleavage, with 90% yield and negligible byproducts formation.

A second advantage is the simpler method provided for the chemical synthesis of the compounds of the invention that requires only six synthetic steps in order to obtain the final compound; moreover, only three purifications by chromatographic column can be sufficient in the method of the invention thereby avoiding the waste of large quantities of purification solvents. Consequently, this synthesis is cheaper and it is therefore possible to synthesize larger quantities at the laboratory level as well as easying the industrial scalability of the process.

Finally, a great advantage of the compound of the present invention, is the possibility of modifying the base molecule with various functional groups in position C₆, thereby allowing the provision of molecules wherein the TLR4 agonist and additional functions may be combined. The different structure of the new compounds of the invention provides a higher stability to the compound and provides position C for functionalization.

In FP11 the C₆ functionalization is challenging: most chemical reagents for functionalization cleave the C₁ phosphate, similarly to what happens during the desilylation. In the case of FP20 compound and derivatives the anomeric phosphate is lacking and the C₆ functionalization is feaseable. This allows the skilled person to customize the compound for specific desired activities by modifying the functional group in C₆, e.g. by increasing its solubility and/or bioavailability, by adding target-specific substituents, by conjugating the molecule with other functional substituents.

Therefore, objects of the invention are:

1. A compound of formula 1

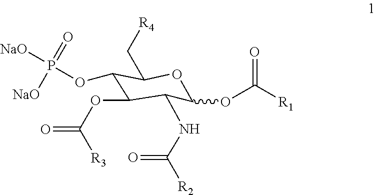

wherein R₁ is a saturated C₅-C₁₅ alkyl chain,

wherein R₂ is a saturated C₅-C₁₅ alkyl chain,

wherein R₃ is a saturated C₅-C₁₅ alkyl chain,

wherein R₄ is any substituent that can be linked by means of a bond between C₆ and a suitable atom and/or any substituent which possesses an oxygen or a nitrogen atom that can bind to C₆. and uses thereof;

A vaccine adjuvant consisting of the compound of formula 1;

A vaccine composition comprising the compound of formula 1, at least one pharmaceutically acceptable carrier and at least one pharmaceutically acceptable immunogenic antigen;

A pharmaceutical composition comprising the compound of formula 1 and at least one pharmaceutically acceptable excipient and/or carrier.

An intermediate of formula 1i

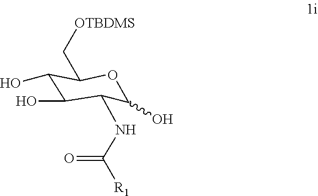

wherein R₁ is a saturated C₅-C₁₅ alkyl chain.

A method for the preparation of an intermediate of formula 1i wherein R₁ is a saturated C₅-C₁₅ alkyl chain

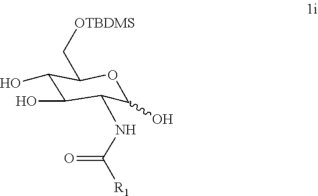

comprising the following steps
1) Selective acylation of the amino group in the C₂ position of glucosamine hydrochloride by reaction with acyl chloride in the presence of sodium bicarbonate.
2) Protection by selective silylation of hydroxyl in position C by reaction with tert-butyldimethylsilyl chloride (TBDMSCI) in the presence of imidazole; A method for the preparation of a compound of formula 1,

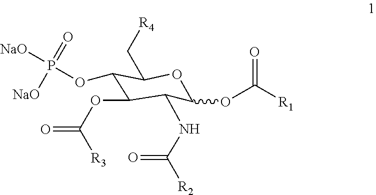

wherein R₁ is a saturated C₅-C₁₅ alkyl chain,

wherein R₂ is a saturated C₅-C₁₅ alkyl chain,

wherein R₃ is a saturated C₅-C₁₅ alkyl chain,

wherein R₄ is any substituent that can be linked by means of a bond between C₆ and a suitable atom and/or any substituent which possesses an oxygen or a nitrogen atom that can bind to C₆., comprising the following steps:
1) Selective acylation of the amino group in the C₂ position of glucosamine hydrochloride by reaction with acyl chloride in the presence of sodium bicarbonate; 2) Protection by selective silylation of hydroxyl in position C by reaction with tert-butyldimethylsilyl chloride (TBDMSCI) in the presence of imidazole, thereby obtaining the intermediate as defined in claim 20;
3) Selective acylation of hydroxyls in positions C₁ and C₃ by reaction with acyl chloride in the presence of triethylamine and N, N-dimethyl aminopyridine (DMAP); 4) Phosphorylation of hydroxyl in the C₄ position by reaction with dibenzyl N, N-diisopropylphospharamidite in the presence of triflate imidazolium, followed by oxidation of phosphite to phosphate via metachloroperbenzoic acid;
5) Deprotection of hydroxyl from silane in position C through the presence of sulfuric acid in catalytic quantities; and
6) Deprotection of phosphate from benzyls in position C₄ and optionally in position C through hydrogenation catalyzed by Palladium on Carbon (Pd/C).

A method for the preparation of a compound of formula X

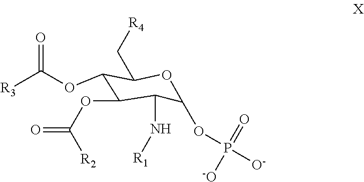

wherein R₁ is a saturated C₅-C₁₅ alkyl chain,

wherein R₂ is a saturated C₅-C₁₅ alkyl chain,

wherein R₃ is a saturated C₅-C₁₅ alkyl chain,

wherein R₄ is OH and wherein each of R₁, R₂ and R₃ is free from —OH substituents in position C₂.
1) Selective acylation of the amino group in the C₂ position of glucosamine hydrochloride by reaction with acyl chloride in the presence of sodium bicarbonate; 2) Protection by selective silylation of hydroxyl in position C by reaction with tert-butyldimethylsilyl chloride (TBDMSCI) in the presence of imidazole, thereby obtaining the intermediateas defined in claim 20;
3) Complete acylation of hydroxyls in positions C₁, C₃ and C₄ by reaction with acyl chloride in the presence of triethylamine and N, N-dimethyl aminopyridine (DMAP);
4) Selective deacylation of position C₁ by reaction with ethylenediamine in presence of acetic acid
5) Phosphorylation of hydroxyl in the C₁ position by reaction with dibenzyl N, N-diisopropylphospharamidite in the presence of triflate imidazolium, followed by oxidation of phosphite to phosphate via metachloroperbenzoic acid;
6) Deprotection of hydroxyl from silane in position C through the presence of sulfuric acid in catalytic quantities;
7) Deprotection of phosphate from benzyls in position C₁ and optionally in position C₆ through hydrogenation catalyzed by Palladium on Carbon (Pd/C).

The use of an intermediate compound as defined in claim 22 for the synthesis of compounds of formula 1,

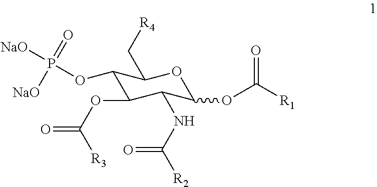

wherein R₁ is a saturated C₅-C₁₅ alkyl chain,

wherein R₂ is a saturated C₅-C₁₅ alkyl chain,

wherein R₃ is a saturated C₅-C₁₅ alkyl chain,

wherein R₄ is any substituent that can be linked by means of a bond between C₆ and a suitable atom and/or any substituent which possesses an oxygen or a nitrogen atom that can bind to C₆.

And,

The use of an intermediate of formula 1i as defined in any one of the embodiments herein disclosed, for the synthesis of compounds of formula X

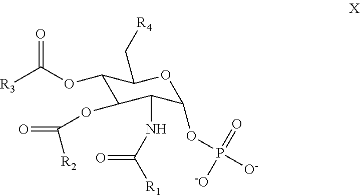

wherein R₁ is a saturated C₅-C₁₅ alkyl chain,

wherein R₂ is a saturated C₅-C₁₅ alkyl chain,

wherein R₃ is a saturated C₅-C₁₅ alkyl chain,

wherein R₄ is OH and wherein each of R₁, R₂ and R₃ is free from —OH substituents in position C₂.

### DETAILED DESCRIPTION OF THE FIGURES

FIG. 1 Activity of FP compounds on TLR4 and TLR2. HEK-BIue™ hTLR4 (A) and HEK-BIue™ TLR2 (B) cells were treated with the indicated concentrations of compounds FP20, FP21, FP22, FP23 and FP24, MPLA, LPS (100 ng/mL) and Pam2CSK4 (1 ng/mL) and incubated for 16-18 hours. The results were normalized with respect to stimulation with LPS alone (A) or Pam2CSK4 (B) and were expressed as a percentage of the mean±SEM of at least three independent experiments. (Treated Vs untreated: * P<0.05; ** P<0.01; *** P<0.001; **** P<0.0001).

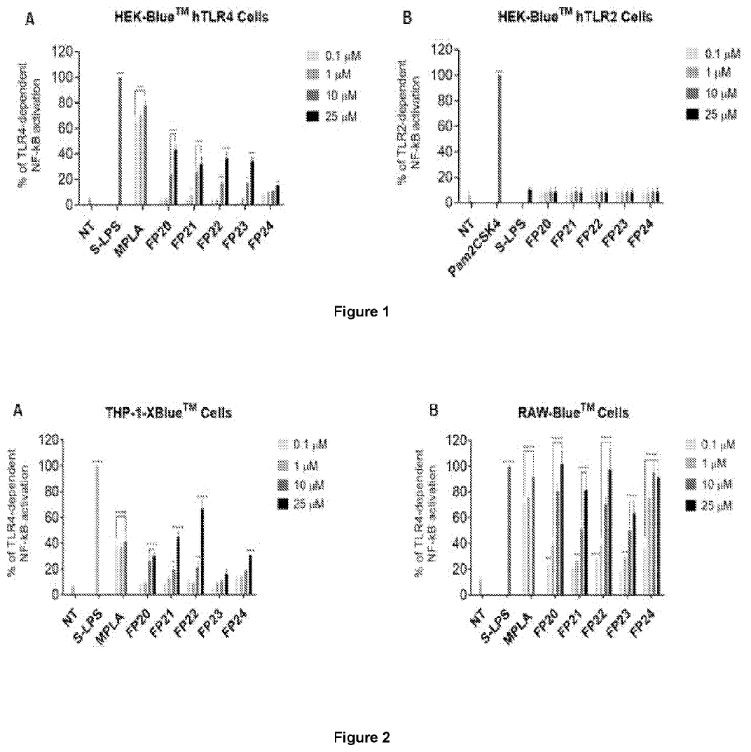

FIG. 2 Activity of FP compounds on human and murine macrophages. THP-1-X BIue™ (A) and RAW-BIue™ (B) cells were treated with the indicated concentrations of compounds FP20, FP21, FP22, FP23 and FP24, MPLA and LPS (100 ng/mL) and incubated for 16-18 hours. The results were normalized with respect to stimulation with LPS alone and were expressed as a percentage of the mean±SEM of at least three independent experiments. (Treated Vs untreated: * P<0.05; ** P<0.01; *** P<0.001; **** P<0.0001).

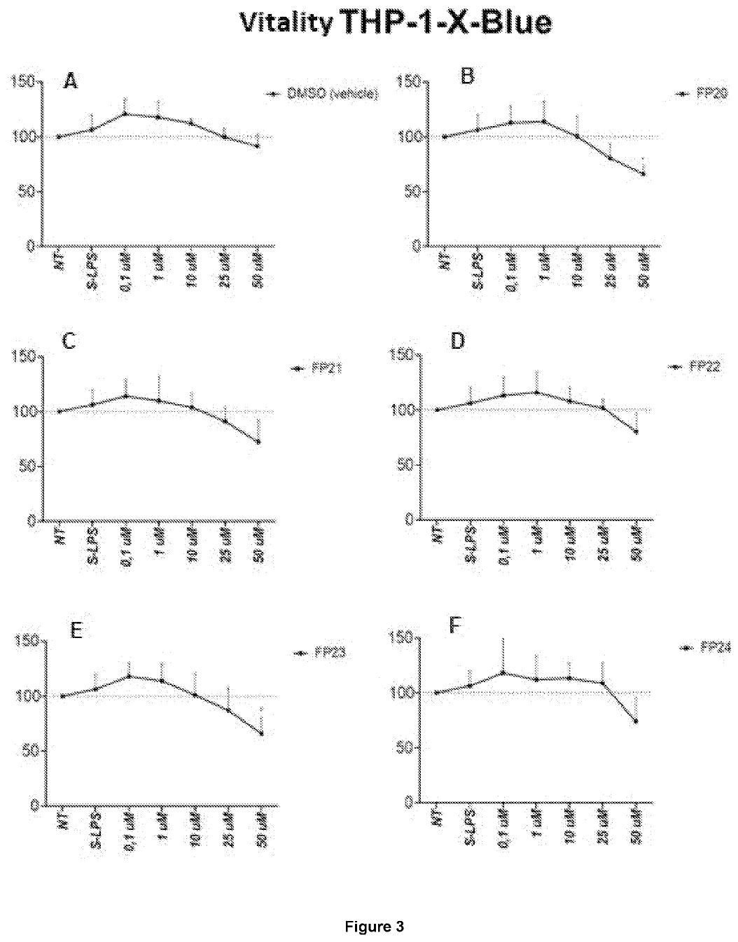

FIG. 3 Cell viability. THP-1 cells differentiated into macrophages were treated with increasing concentrations of FP20, FP21, FP22, FP23 and FP24 (0.1-50 μM) and LPS (100 ng/mL). The vehicle (DMSO) at the same concentrations (0.1-50 μM) was inserted to evaluate the toxicity of the solvent. Data were normalized to (untreated) control and expressed as a percentage of the mean±SEM of at least three independent experiments (Treated Vs untreated: * P<0.05; ** P<0.01; *** P<0.001; **** P<0.0001).

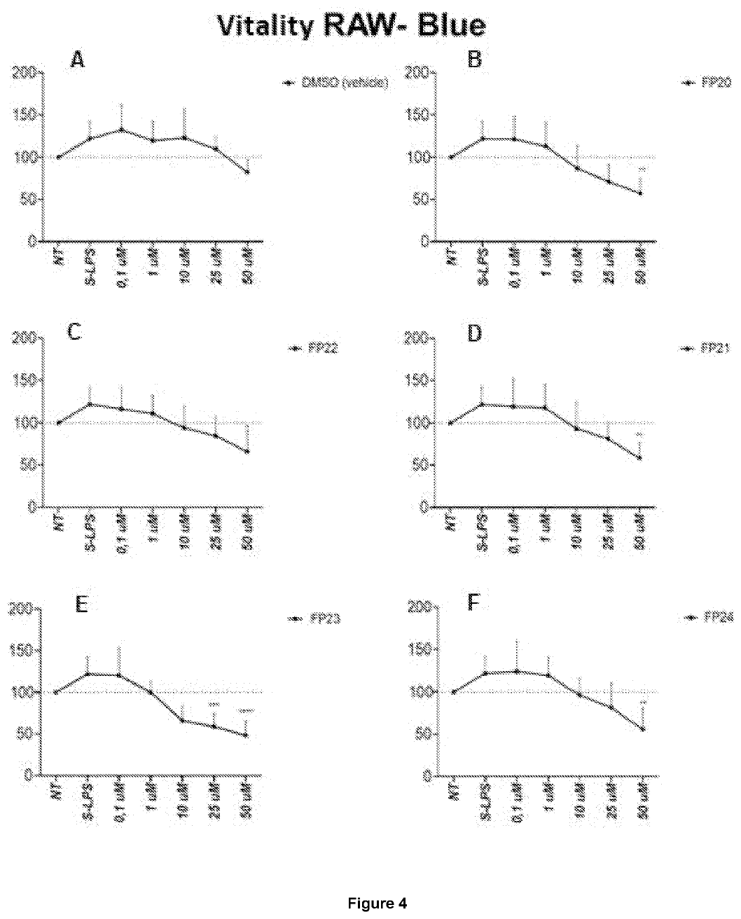

FIG. 4 Cell viability. RAW-Blue cells were treated with increasing concentrations of FP20, FP21, FP22, FP23 and FP24 (0.1, 1, 10, 25, 50 μM) and LPS (100 ng/mL). The vehicle (DMSO) at the same concentrations (0.1, 1, 10, 25, 50 μM) was inserted to evaluate the toxicity of the solvent. Data were normalized to (untreated) control and expressed as a percentage of the mean±SEM of at least three independent experiments. (Treated Vs untreated: * P<0.05; ** P<0.01; *** P<0.001; **** P<0.0001)

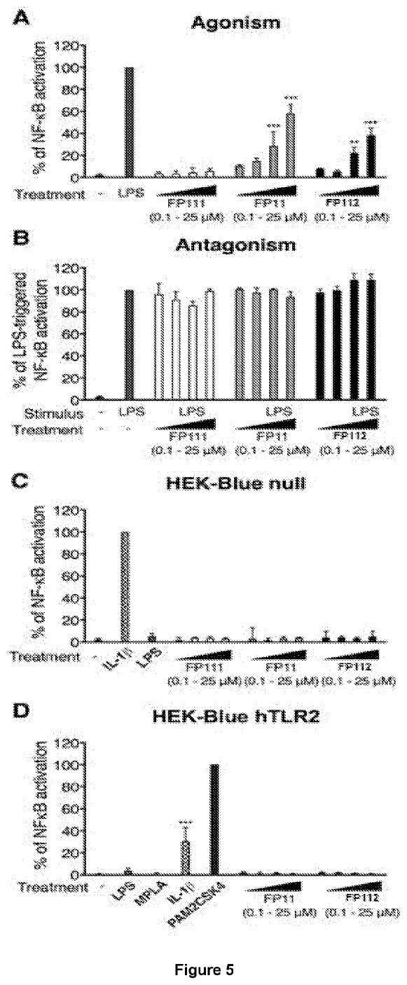

FIG. 5 HEK-Blue hTLR4, HEK-Blue Null and HEK-Blue hTLR2 cells were treated as indicated and incubated for 18 h. Supernatants were collected and SEAP levels were quantified by QUANTI-blue method. Data were normal-ized to stimulation with S-LPS (A, B), IL-1β (C) or PAM2CSK4 (D) and expressed as the mean percentage ±SD of three independent experiments. (Treated versus untreated: **p<0.01; ***p<0.001).

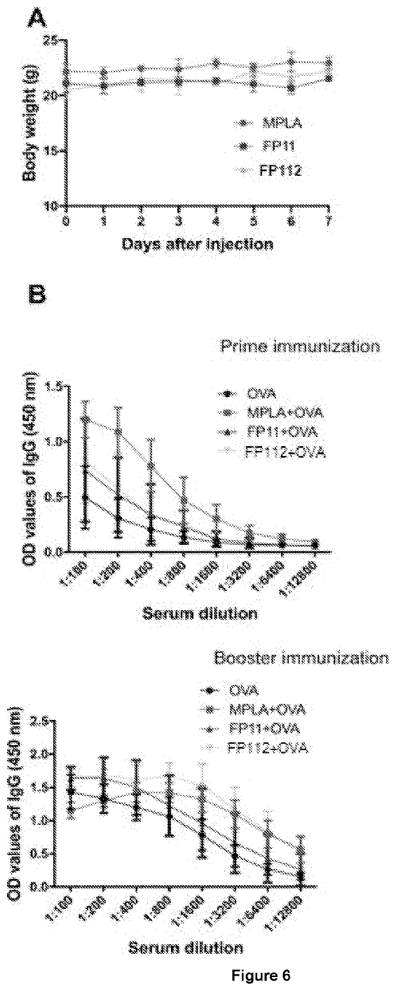

FIG. 6 A) Body weight of mice over 7 days after administration of adjuvants (n=4 per treatment). B) Antibody response to OVA immunization using MPLA, FP112 and FP11 as adjuvants after prime (22 days post immunization) and booster immunization (19 days later) (n=8 per treatment). For statistical comparisons the area under each curve was examined by Brown-Forsythe and Welch One-way ANOVA tests with an alpha of 0.05.

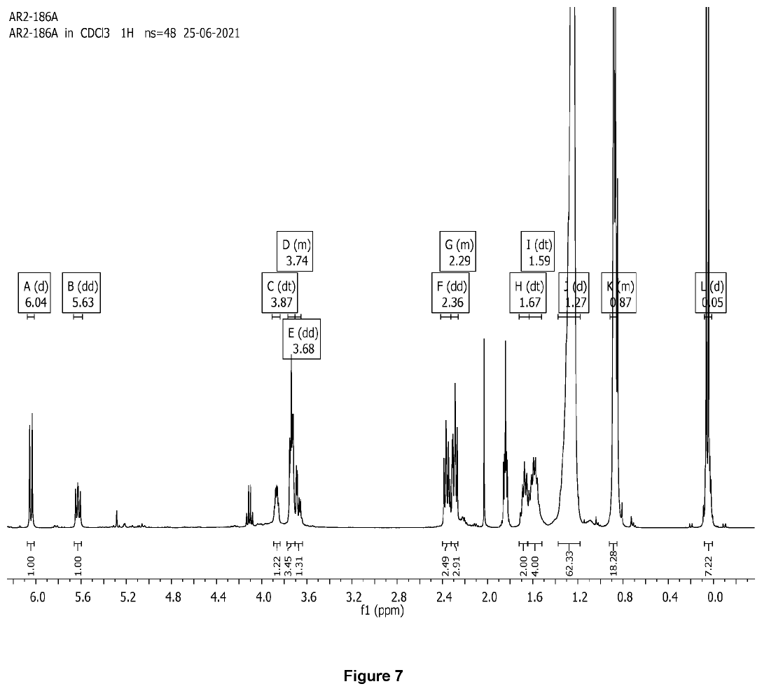

FIG. 7 ¹H NMR of compound 25 (Impurity 1 of step 6 in FP11 Synthesis), in which is possible to observe the cleavage of the phosphate in C-1 by the multiplicity (d) of the signal at 6.04 ppm. In the presence of a phosphate in C-1, H-1 would have a dd multiplicity due to the H—P coupling.

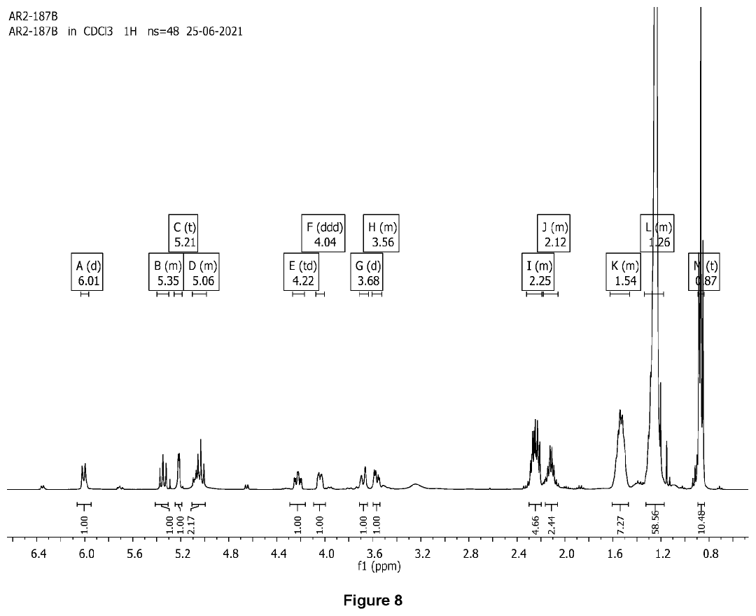

FIG. 8 ¹H NMR of compound 26 (Impurity 2 of step 6 in FP11 Synthesis), in which is possible to observe the cleavage of the phosphate in C-1 by the multiplicity (d) of the signal at 6.01 ppm. In the presence of a phosphate in C-1, H-1 would have a dd multiplicity due to the H—P coupling. In this case, also the silane has been cleaved, as observed by the lack of their signals at 0 ppm and 0.85 ppm.

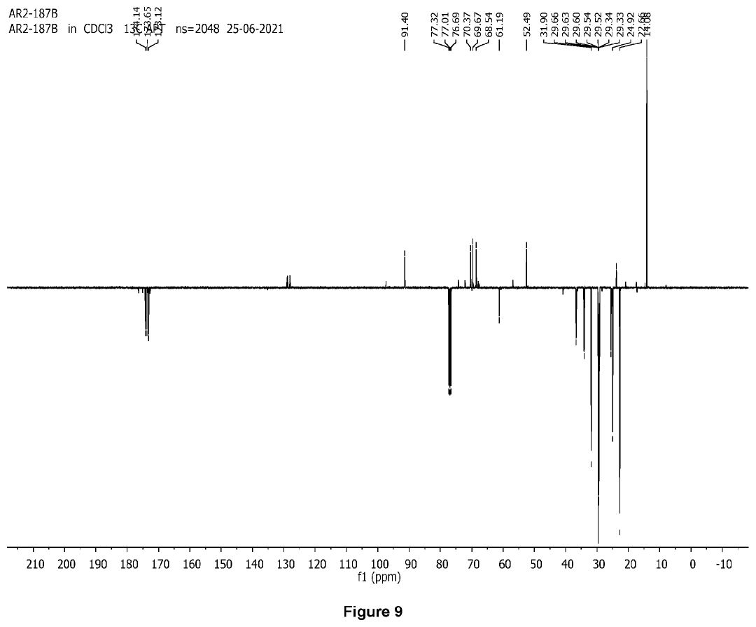

FIG. 9 ¹³C NMR of compound 26 (Impurity 2 of reaction 6 in FP11 Synthesis), which confirms the structure of compound 26 as observed in FIG. 8.

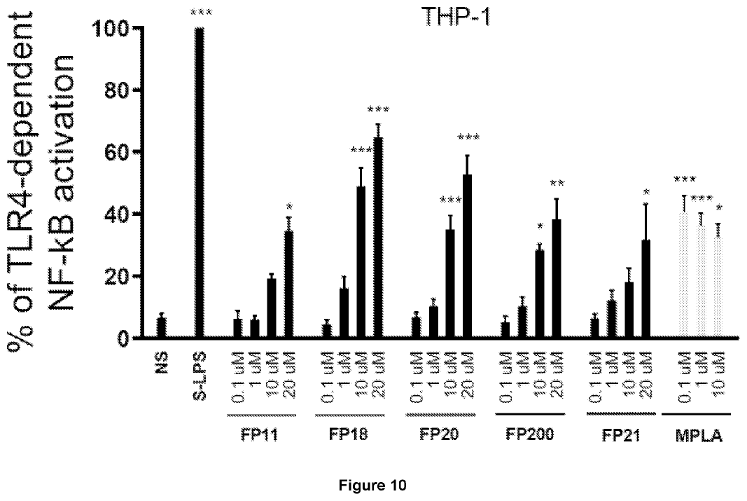

FIG. 10 Activity of FP200 diphosphate compounds on human macrophages. Differentiated THP-1-X Blue™ cells were treated with the indicated concentrations of compounds FP11, FP112, FP20, FP200, FP21, MPLA and LPS (100 ng/mL) and incubated for 16-18 hours. The results were normalized with respect to stimulation with LPS alone and were expressed as a percentage of the mean±SEM of at least three independent experiments. (Treated Vs untreated: * P<0.05; ** P<0.01; *** P<0.001; **** P<0.0001).

FIG. 11—Activity of FP207 on human macrophages

Differentiated THP1-XBlue™ were treated with the indicated concentrations of FP20, FP207, MPLA and LPS (100 ng/mL) and incubated for 16-18 hours. The results were normalized with respect to stimulation with LPS alone and were expressed as a percentage of the mean±SEM of at least three independent experiments. (Treated Vs untreated: * P<0.05; ** P<0.01; *** P<0.001; **** P<0.0001).

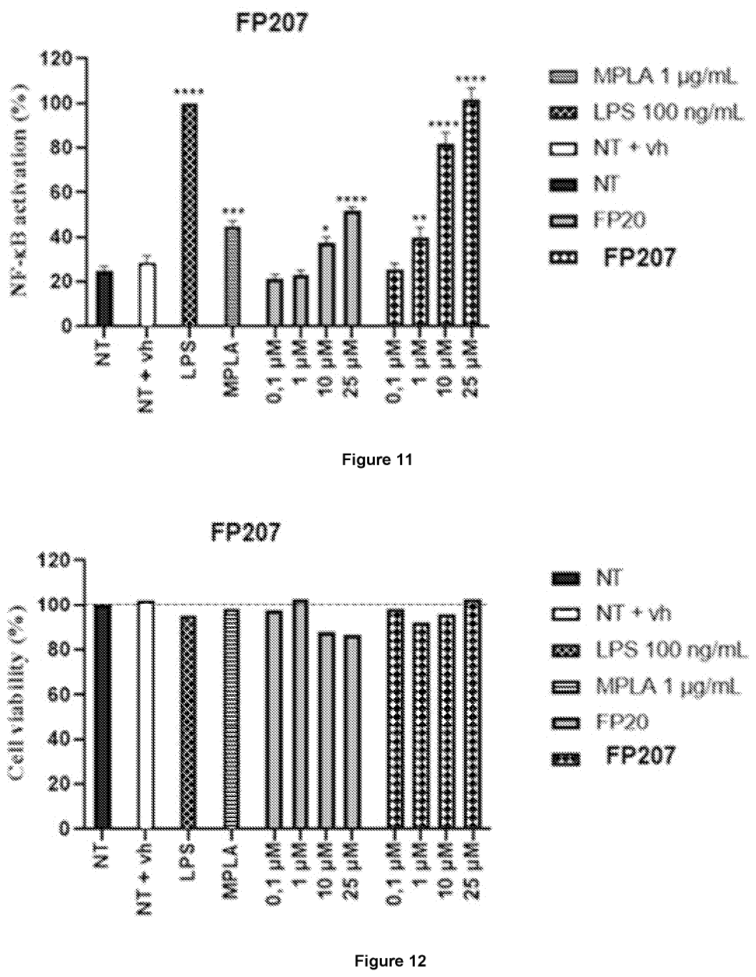

FIG. 12—Cell Viability

Differentiated THP1-XBlue™ cells were treated with increasing concentrations of FP20 and FP207 (0.1-25 μM), MPLA and LPS (100 ng/mL). Data were normalized to (untreated) control and expressed as a percentage of the mean±SEM of at least three independent experiments.

### GLOSSARY

In the present description, the term “TLR4 receptor agonist” denotes a compound that selectively binds to the TLR4 receptor inducing a conformational change of said receptor, in turn generating an intracellular stimulation by triggering a response similar to that induced by the natural ligand of said receptor. In the case of TLR4, the substances described as agonists bind to co-receptor MD-2, in turn non-covalently bound to TLR4, thereby generating the receptorial complex (TLR4/MD-2/agonist)₂, which from the cell surface initiates a signal cascade leading to activation of nuclear transcription factors and synthesis of pro-inflammatory cytokines (mainly TNF-α and various interleukin types).

In the present description, the compound identified as FP112 refers to a compound having the formula represented below

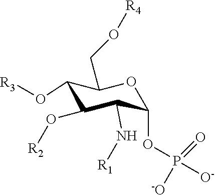

wherein R₁=R₂=R₃=C═OC₁₁H₂₃ and R₄=H, disclosed as FP112 in WO2019/092572.

In the present description, the bond in C₁ as represented in Formula 1 below has the meaning commonly intended in organic chemistry

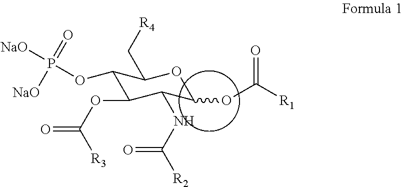

and indicates that the the compound can be either in the a or in the p anomer conformation.

In the present description, the bond in C₁ as represented in Formula 1a below has the meaning commonly intended in organic chemistry.

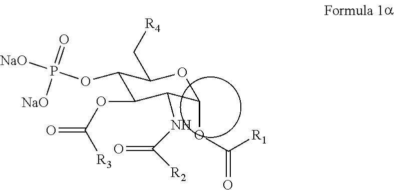

and indicates that the the compound is in the a anomer conformation.

In the present description, the bond in C₁ as represented in Formula 1a below has the meaning commonly intended in organic chemistry.

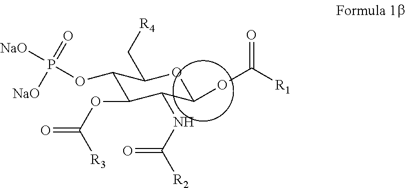

and indicates that the the compound is in the p anomer conformation.

In the present description, the term “catalytic quantities” means an amount or a concentration of a substance used in a chemical reaction such as to obtain a catalytic effect. In particular, in the description the term “catalytic quantities” can be substituted by “a range from 0.5% to 1% of volume/volume concentration”.

### DETAILED DESCRIPTION OF THE INVENTION

The present invention relates to a compound of formula 1

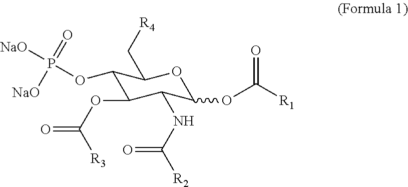

wherein R₁ is a saturated C₅-C₁₅ alkyl chain,

wherein R₂ is a saturated C₅-C₁₅ alkyl chain,

wherein R₃ is a saturated C₅-C₁₅ alkyl chain,

wherein R₄ is any substituent known by the skilled person that can be linked by means of a bond between C₆ and a suitable atom and/or any substituent which possesses an oxygen or a nitrogen atom that can bind to C₆.

Therefore, according to the present description, each alkyl chain, R₁, R₂, or R₃, can be a C₅, C₆, C₇, C₈, C₉, C₁₀, C₁₁, C₁₂, C₁₃, C₁₄, C₁₅ alkyl chain.

According to the description each of R₁, R₂, and R₃ can be a different or identical alkyl chain as defined above.

In one embodiment of the invention, at least two of R₁, R₂, and R₃ are identical.

According to an embodiment of the invention said R₁, R₂, and R₃ chains are free from —OH substituents on position C₂. The absence of hydroxyls in position C₂ advantageously allows a shorter and more efficient synthetic route, eliminating various protection and de-protection steps of the hydroxyl groups thereby, reducing the costs of synthesis and making this synthetic process scalable and industrializable for drugs production.

According to another embodiment of the invention, said R₁, R₂, and R₃ chains are free from any substitutent.

As described above, the R₄ chain in position C can be any functionalization substituent of interest provided that the TLR4 agonist activity is not disrupted.

According to a non limiting example, the R₄ can be an hydroxyl group (OH), a phosphate group (PO₄²⁻), an azide group (N₃), an amine group (NH₂), an acyl group (O(C═O)R), an alkyl group (OR) or a glycosyl group.

The suitability of R₄ in position C to functionalization is an extremely advantageous feature of the compounds of the invention. As described above, it is indeed possible to provide molecules in which the agonist function of TLR4 and any other function of interest depending on the selected substituent of R₄ may be combined. For example, it is surprisingly possible to obtain an increase in solubility and bioavailability using a phosphate group (PO₄²⁻). In fact, according to the teachings in the art (WO2019/092572) the presence of two phosphate groups in the agonists described therein resulted in the absence of TLR4 agonist activity. On the contrary, the new compounds of the invention surprisingly retain the TRL4 agonist activity also with a second phosphate group thereby improving the solubility of the compound itself. As known by the skilled person, improved solubility is an important advantage as it ameliorates the delivery, the bioavailability of the compound and the stability of a composition comprising it. Hence, the simple presence of an additional phosphate in C₆, can significatively improve the efficiency of a pharmaceutical or vaccine composition.

Another important advantage of the possible functionalization in C of the compounds of the invention is that, when used as a vaccine adjuvant it is also possible to conjugate it to an antigen or to an antigenic epitope, or to conjugate it to a different adjuvant to improve and expand its activity.

In addition, the compound can be conjugated to a target-specific molecule thereby improving its delivery at a site of preference.

Furthermore, when used in an anti-tumoral composition, the compound of the invention may be functionalized by linking it to additional, different, drugs in order to improve its effectiveness.

By way of example, it is possible to use substituents free of hydrogen atoms capable of forming hydrogen bonds in order to improve the lipophilic effect, or substituents characterized by the presence of hydrogen atoms capable of forming hydrogen bonds in order to improve solubility in water, which is inversely related to lipophilicity.

A further advantage deriving from the possibility of exploiting a large variety of substituents as R₄, is the steric effect that can derive for example by using a substituent which tends to limit the free rotation around simple bonds, and therefore to reduce the number of energetically accessible conformations.

When the bioactive one is present among these conformations, the “stiffening” effect can increase the affinity of the molecule for the receptor.

Another important example may be represented by the functionalization with a linker in position C₆. A non limiting example of suitable linkers si represented by a disulfide (R—S—S—R′), an hydrazone (R′R″C═N—NH₂), a peptide or a thioeters (R′—S—R″) or the like.

Linkers such as the ones described above allow the preparation of an Antibody-Drug-Conjugate (ADC) which is a complex molecule comprising an antibody linked to a biologically active anticancer payload or drug (such as the compounds of the invention), allowing to obtain a combination effect between the antibody and the compound of the present invention.

A further interesting instance is the possibility to insert a glycosyl group in C₆, as the resulting compound would have two advantages: an improved water solubility due to the presence of the hydrophilic glycosyl group and theoretically a better affinity for the receptor due to the mimicking of the core portion of LPS.

Suitable substituents depending on the desired property are known to the skilled person.

According to the present invention, the compound of formula 1 may be an α-anomer having formula 1a or an β-anomer having formula 1β.

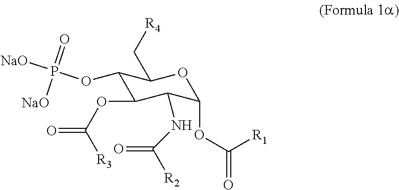

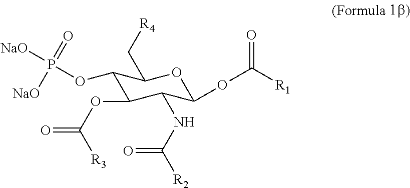

According to some possible non-limiting embodiments, the compound of formula 1 may be selected from compounds in the form of α-anomer or β-anomer of compounds of Formula 1, wherein
R₁=R₂=R₃=C₁₁H₂₃ and R₄=OH, or
R₁=R₃=C₁₃H₂₇; R₂=C₁₁H₂₃ and R₄=OH, or
R₁=R₂=R₃=C₉H₁₉ and R₄=OH, or
R₁=R₂=R₃=C₁₃H₂₇ and R₄=OH, or
R₁=R₃=C₉H₁₉; R₂=C₁₁H₂₃ and R₄=OH, or
R₁=R₂=R₃=C₁₁H₂₃ and R₄=PO₄²⁻, or
R₁=R₂=R₃=C₉H₁₉ and R₄=PO₄²⁻ or
R₁=R₂=R₃=C₁₃H₂₇ and R₄=PO₄²⁻, or
R₁=R₂=R₃=C₁₁H₂₃ and R₄=OC₃H₇, or
R₁=R₂=R₃=C₁₁H₂₃ and R₄=O(C═O)C₆H₈(OH)₃, or
R₁=R₂=R₃=C₁₁H₂₃ and R₄=NH₂
R₁=R₂=R₃=C₁₁H₂₃ and R₄=O(C═O)CCH₃(CH₂OH)₂
R₁=R₂=R₃=C₁₁H₂₃and R₄=OCH(CHOH)₃CH(CH₃)O
R₁=R₂=R₃=C₁₁H₂₃and R₄=OCH(CHOH)₃CH(CH₂OH)O
R₁=R₂=R₃=C₁₁H₂₃ and R₄=OCH(CHOH)₃(CH₂)O.
   In a preferred embodiment the compounds are β-anomer of compounds of Formula 1 such as:

Compound FP20: with R₁=R₂=R₃=C₁₁H₂₃ R₄=OH

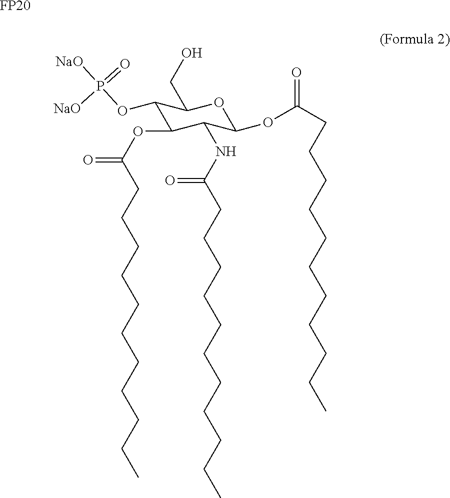

compound FP21: with R₁=R₃=C₁₃H₂₇ R₂=C₁₁H₂₃ R₄=OH

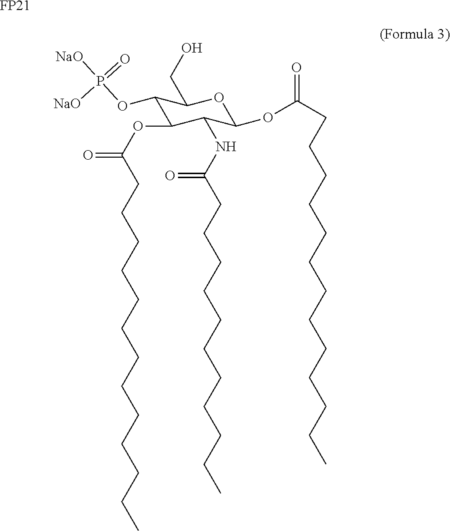

compound FP22: with R₁=R₂=R₃=C₉H₁₉ R₄=OH

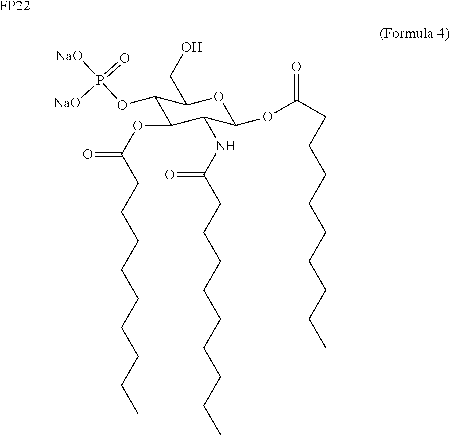

compound FP23: with R₁=R₂=R₃=C₁₃H₂₇ R₄=OH

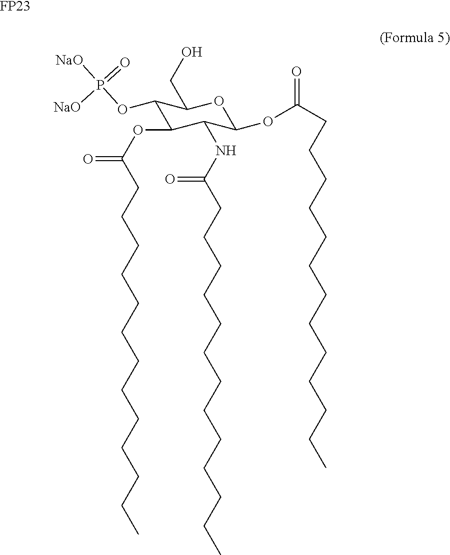

compound FP24: with R₁=R₃=CH₁₉ R₂=C₁₁H₂₃ R₄=OH

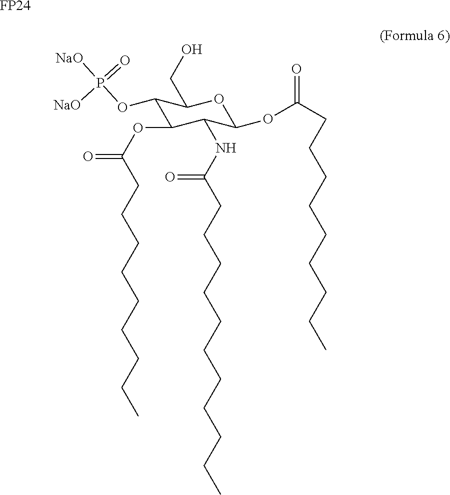

compound FP200: with R₁=R₂=R₃=C˜H₂₃ R₄=PO₄²⁻

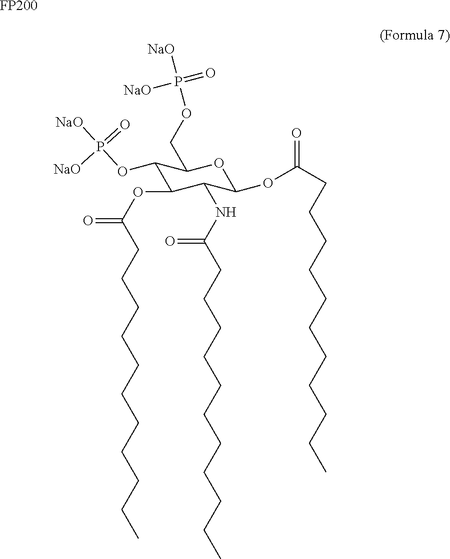

compound FP202: with R₁=R₂=R₃=C₉H₁₉ R₄=PO₄²⁻

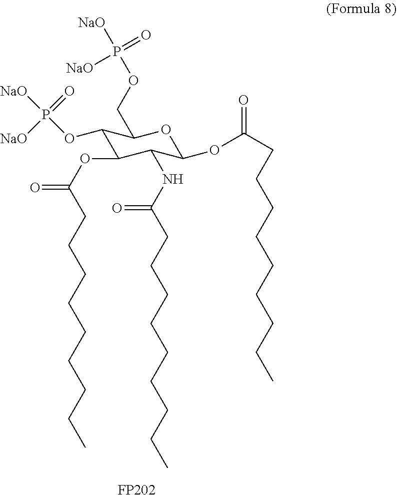

compound FP203: with R₁=R₂=R₃=C₁₃H₂₇ R₄=PO₄²⁻

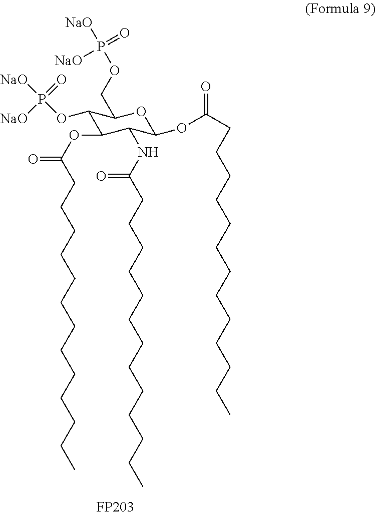

Further embodiments according to the present invention, wherein the compound of formula 1 having additional substituent on position C may be selected from the following ones:
compound FP204: with R₁=R=R₃=C₁₁H₂₃ R₄=OC₃H₇

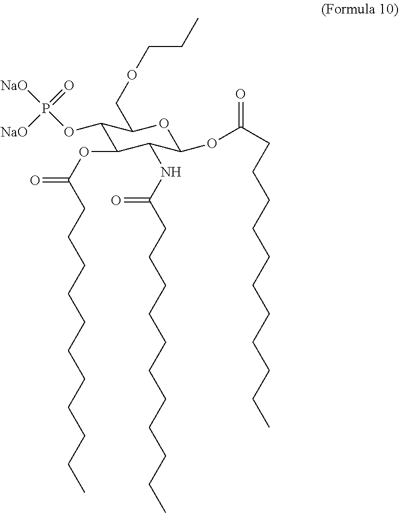

compound FP205: with R₁=R₂=R₃=C₁₁H₂₃ R₄=O(C═O)C₆H₈(OH)₃

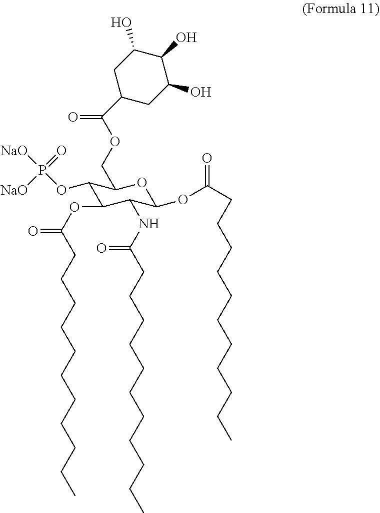

compound FP206: with R₁=R₂=R₃=C₁₁H-23 R₄=NH₂

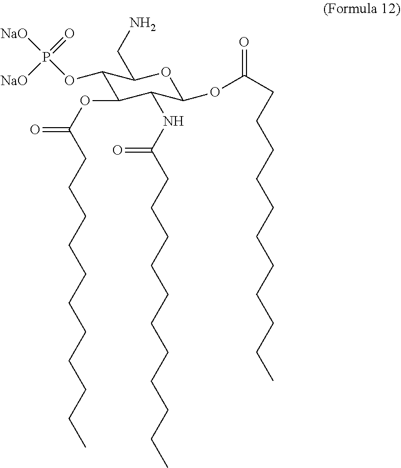

compound FP207: with R₁=R₂=R₃=C₁₁H₂₃ R₄=O(C═O)CCH₃(CH₂OH)₂

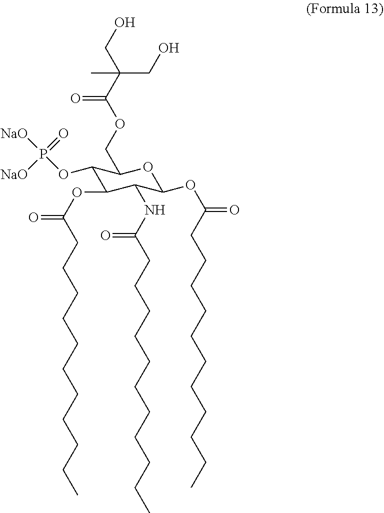

compound FP20Rha: with R₁=R₂=R₃=C₁₁H₂₃ R₄=OCH(CHOH)₃CH(CH₃)O

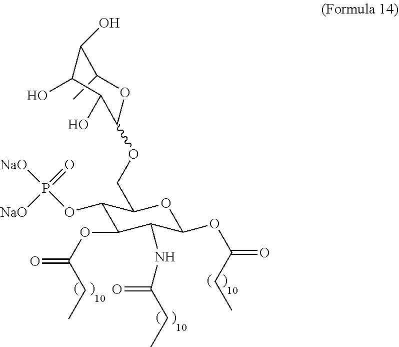

compound FP20GIc: with R₁=R₂=R₃=C₁₁H₂₃ R₄=OCH(CHOH)₃CH(CH₂OH)O

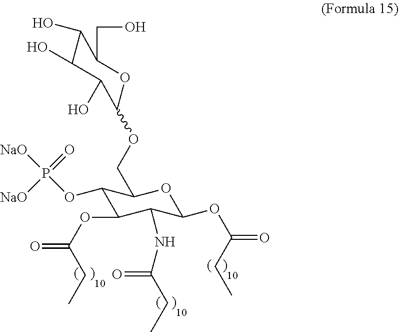

Compound FP20Man: with R₁=R₂=R₃=C₁₁H₂₃ R₄=OCH(CHOH)₃CH(CH₂OH)O

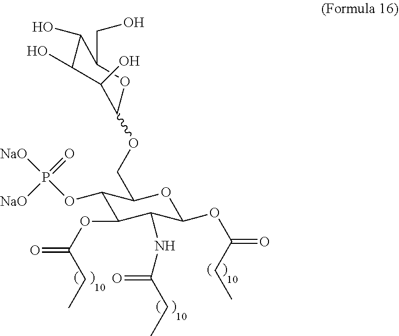

Compound FP20Gal-α: with R₁=R₂=R₃=C₁₁H₂₃ R₄=OCH(CHOH)₃CH(CH₂OH)O

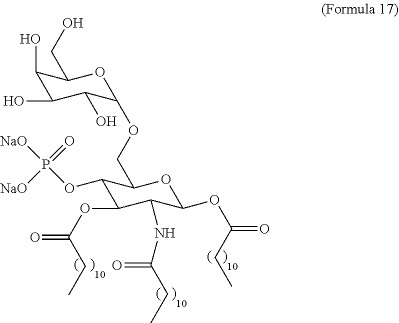

Compound FP20Gal-β: with R₁=R₂=R₃=C₁₁H₂₃ R₄=OCH(CHOH)₃CH(CH₂OH)O

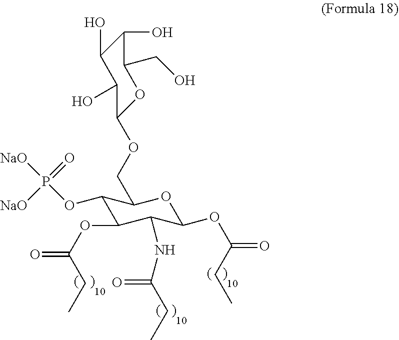

Compound FP20Lyx: with R₁=R₂=R₃=C₁₁H₂₃ R₄=OCH(CHOH)₃CH₂O

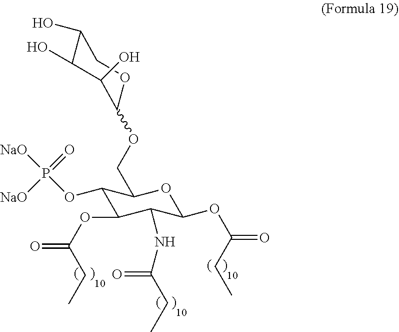

Compounds of the present invention having formula 14, 15, 16 or 19 are mixtures of anomers (diastereoisomers) of the sugars bound to C₆.

Compounds of the present invention having formula 17 or 18 are respectively the pure alpha and beta anomers of glucose bound in C₆.

In one embodiment, the compound having formula 2 is preferred.

In the present description, such a compound is also referred as compound FP20, wherein R₁, R₂ and R₃ are —C₁₁H₂₃ and R₄ is —OH.

Data reported in the Examples section show the peculiar advantageous features of the above-indicated compounds.

According to the present description, and on the basis of experimental data obtained, it is evident that the compounds as described and claimed are effective agonists of TLR4 receptor. By “agonist of a receptor” (receptor agonist) it is meant as is commonly defined in the literature, i.e., a substance able to bind a specific receptor in the binding site for the endogenous ligand. Therefore, as the name suggests, the former competes with the latter for the binding with said site.

Following binding with the natural ligand, the receptor encounters conformational changes that mediate its biological activity at cell level. Agonists are molecules having inherent activity able to mimic ligand effects. When binding to the receptor, they cause conformational changes of an extent similar to those caused by binding with the endogenous ligand.

In the case of the present description, each agonist disclosed and claimed is an agonist selective for the TLR4 receptor.

Given the technical features observed for compounds of formula (1) as defined in the present description and in the claims, said compounds are useful as active principles or as adjuvants in the treatment of diseases benefiting from a TLR4 receptor activation, i.e., in diseases in which an activation of the immune system, particularly of the innate activity, has a therapeutic or prophylactic effect.

Therefore, among diseases requiring or benefiting from a TLR4 receptor activation, are included all the diseases whose treatment or whose prevention are improved by TLR4 receptor activation and by the innate immune response triggered by the activation of said receptor.

A non-limiting example of such diseases is represented by tumours, allergies, infectious diseases such as viral infectious, cardiovascular diseases, obesity-dependent metabolic diseases, neuronal degeneration, apoptosis, autoimmune disorders, bacterial infections autoimmune diseases. An example of autoimmune diseases is represented by IBD, Chron's disease or rheumatoid arthritis.

The compound of formula 2, herein also identified as compound FP20, can be compared with the compound of formula FP112 having the formula represented below

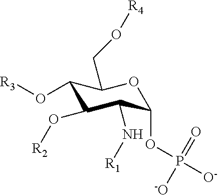

wherein R₁=R₂=R₃=C═OC₁₁H₂₃ R₄=H, disclosed in WO2019/092572, named therein as FP112, due to the fact that they have the same R₁, R₂, R₃ and R₄ substituents, but the phosphate group is on position C₁ for FP112 and on position C₄ for FP20.

Experiments in vitro conducted for FP112 on cells Hek-Blue, Raw-Blue and THP-1 briefly reported in the examples below, demonstrated a low toxicity and a good pro-inflammatory activy at a concentration of 10 μM. Furthermore, experiments in vivo conducted with FP112 to test its tolerability and effectiveness as vaccine adjuvant, demonstrated no collateral damage from the administration of 10 μg of FP112, and an effectiveness as vaccine adjuvant comparable to that of MPLA.Data reported in the Examples section show that for experiments conducted on Hek-Blue, Raw-Blue and THP-1 cells, show that the activity of the compounds of the invention is comparable with the activity of the agonists compounds disclosed in WO2019/092572 which proved to be also non cytotoxic and effective as vaccine adjuvants in vivo.

Additionally, the compounds of the present invention are improved with respect to the compounds disclosed in WO2019/092572 due to the re-location of the phosphate group in C₁ in the prior art, in position C₄ in the present invention, thereby allowing the positioning of the alkyl chains in C₁ in the compounds of the present invention rather than in C₄, which resulted in a enchanced activity of the substituents in C₆. In fact, WO2019/092572 discloses in Experiment 2 that a compound named therein as FP111, having two phosphate groups: one in position C₁ and the other one in position C proved completely inactive as TLR4 agonist in a test conducted on HEK-Blue™ hTLR4 cells. In WO2019/092572 it was speculated that the absence of activity could be due to the presence of two phosphates in the molecule and that the number of phosphates in the molecule had to be no more than 1 in order to maintain their TLR4 agonist activity.

Surprisingly, the Authors of the present invention discovered that the compounds disclosed and claimed in the present application, such as, by way of example compounds of formula 7, 8 and 9, bearing two phosphate group, one in position C₄ and another one in position C₆, surprisingly maintained the TLR4 agonist activity in the same tests in which compound FP111 disclosed in WO2019/092572 resulted completely inactive. This finding was unexpected and proves a relevant advantage of the compounds of the invention over the prior art as it demonstrates that the position of phosphate groups in a triacylated monophosphoryl glucosamine core, can significantly vary the activity of such compound.

The invention therefore also provides the compound of formula 1 in any one of the embodiments disclosed in the description or in the claims as vaccine adjuvant.

The relevance of immune response adjuvants in vaccine composition is known. Vaccine adjuvants, in fact, substantially increase vaccine effectiveness and development of immunity, in the treated subject, toward antigens present in the vaccine.

Therefore, object of the present invention is also a vaccine composition comprising the compound of formula 1 as defined in any one of the embodiments in the description or in the claims or a mixture thereof.

The vaccine composition according to the invention can therefore comprise the compound of formula 1 as described herein or a mixture thereof, in any one of the above-listed embodiments, at least one pharmaceutically acceptable carrier and at least one antigenic compound able to induce a desired immune response, such as an immunogenic antigen.

Suitable vaccine carriers are known to the skilled person.

The pharmaceutical carrier may be selected to assist release of the antigen component(s) over an extended period of time from the composition. The carrier may include a water-soluble or water-insoluble substance.

A water-soluble substance is a substance which plays a role in controlling infiltration of water into the interstices of the drug dispersion.

One water-soluble substance, or a combination of two or more water-soluble substances may be used.

The water-soluble substance specifically may be selected from one or more of the groups consisting of synthetic polymers (eg. polyethylene glycol, polyethylene polypropylene glycol), sugars (eg. sucrose, mannitol, glucose, sodium chondroitin sulfate), polysaccharides (e.g. dextran), amino acids (eg. glycine and alanine), mineral salts (eg. sodium chloride), organic salts (eg. sodium citrate) and proteins (eg. gelatin and collagen and mixtures thereof).

In addition, when the water-soluble substance is an amphiphilic substance, which dissolves in both an organic solvent and water, it has an effect of controlling the release of, for example, a lipophilic drug by altering the solubility thereof. An amphiphilic substance includes, but not limited to, one or more selected from the group consisting of polyethylene glycol or a derivative thereof, polyoxyethylene polyoxypropylene glycol or a derivative thereof, fatty acid ester and sodium alkylsulfate of sugars, and more specifically, polyethylene glycol, polyoxy stearate 40, polyoxyethylenepolyoxypropylene-glycol, polyoxyethylene-polyoxypropylene-glycol, polyoxyethylene-polyoxypropylene-glycol, sucrose esters of fatty acids, sodium lauryl sulfate, sodium oleate, sodium chloride, sodium desoxycholic acid (or sodium deoxycholic acid (DCA)) of which mean molecular weights are more than 1500.

In addition, the water-soluble substance may include a substance selected from one or more of the groups consisting of drugs, peptides, proteins, glycoproteins, polysaccharides, or an antigenic substance used as vaccines.

A water-insoluble carrier, when present, may include a substance which plays a role in controlling infiltration of water into the interstices of the drug dispersion. One water-insoluble substance, or a combination of two or more water-insoluble substances may be used.

The water-insoluble substance specifically may be selected from one or more of the groups of water insoluble polymers, resins and latexes including water-insoluble acrylates, methacrylates and other carboxy polymers, waxes, lipids including phospholipids and lipoproteins.

The skilled person knows the amount of carrier and optional further eccipients commonly used in a pharmaceutical or in a vaccine composition.

In an embodiment, the pharmaceutical carrier may constitute from approximately 1% to 20% by weight, preferably approximately 10% to 20% by weight, based on the total weight of the vaccine composition.

The composition according to the invention may comprise one of the compounds as defined and claimed herein, or a mixture thereof.

The composition of the invention may be prepared in the form of a single mixture of adjuvant and antigen or in the form of different mixtures for a concomitant or sequential administration of the components.

In a particular embodiment, the compound of formula 1 described in the present invention is the sole adjuvant present in the vaccine composition.

The present invention also relates to a pharmaceutical composition, comprising a compound of formula 1 described in any one of the embodiments provided in the description or in the claims or a mixture thereof and at least one pharmaceutically acceptable excipient and/or carrier.

The composition may further comprise one or more additional therapeutically active principle.

Said pharmaceutical composition can also be formulated in the form of an association of a plurality of active principles.

The pharmaceutical composition of the invention may comprise as sole active principle one or more compounds of formula 1 according to any one of the embodiments provided in the description or in the claims, or could also comprise additional active principles, such as anti-tumour active principles, kinase inhibitors, cytotoxic compounds and at least one pharmaceutically acceptable carrier or excipient.

The pharmaceutical composition may be formulated for oral, parenteral, nasal, aerosol, sublingual, rectal, vaginal, topical, endovenous or systemic administration. suitable conventional carriers and/or excipients for suspension, emulsion, ointment, cream, spray, granulate, powder, solution, capsule, pill, tablet, lyophilized product, lozenge, aerosol, nebulization, injection, or others can be selected by the person skilled in the art.

Further object of the present invention is the pharmaceutical composition according to any one of the embodiments herein disclosed for use in the treatment, or as an adjuvant in the treatment, of diseases that require or benefit from an immunostimulation by activating the TLR4 receptor.

Diseases that require or benefit from an immunostimulation by activating the TLR4 recepto, are known in the art and comprise cancer, allergies, infectious diseases, cardiovascular diseases, obesity-dependent metabolic diseases, neuronal degeneration, apoptotic diseases, autoimmune disorders, viral infections, bacterial infections, autoimmune diseases. An example of autoimmune diseases is represented by IBD, Chron's disease or rheumatoid arthritis.

According to the invention, the composition may comprise 0.01 to 50 mg of compound of the invention or of a mixture thereof per daily dosage, by way of example 0.01 to 50 mg of substance per Kg of body weight (test on animals).

The invention also provides a new method for the synthesis of compounds of formula 1 as herein defined as well as for the synthesis of the compounds disclosed in WO2019/092572 of formula X

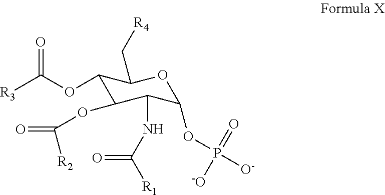

wherein R₁ is a saturated C₇-C₁₅ alkyl chain,

wherein R₂ is a saturated C₇-C₁₅ alkyl chain,

wherein R₃ is a saturated C₇-C₁₅ alkyl chain,

wherein R₄ is OH and wherein each of R₁, R₂ and R₃ is free from —OH substituents in position C₂, and for the synthesis of an intermediate of of formula 1i

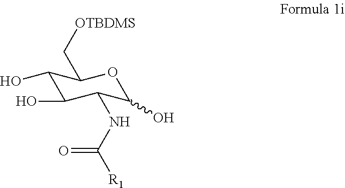

wherein R₁ is a saturated C₄-C₁₅ alkyl chain. According ot an embodiment of the invention, said R₁ is free from any substituent

The compounds of formula 1, as well as the compounds disclosed in WO2019/092572, can be synthesized in a simpler and industrially scalable way compared to the synthesis methods knonw in the art for the compounds of WO2019/092572 as well as for SDZ MRL953. The latter requires the insertion of three acyl chains of (R)-3-hydroxymyristic acid. The optically pure compound (R-enantiomer) is not commercially available, as only the racemic mixture is marketed. Moreover, (R)-3-hydroxymyristic acid requires a reaction of protection of the hydroxyl group in 3 position prior to the condensation reaction with the sugar. The method disclosed in WO2019/092572 for the synthesis of compounds of formula X as defined above, although already simplified with respect to the synthesis method disclosed for SDZ MRL953, due to the absence of substituents on the acyl chains, still comprises 10 steps; a number of purifications by chromatographic column and some critical steps, such as the formation of a low molecular weight azide. Additionally, the method disclosed in WO2019/092572 has an extremely low yield (about 8-9%), which makes the whole process uneconomic.

The compounds provided in the present invention exhibit biological activities comparable to, if not even better than, the compounds of the art and can be synthesized in a much simpler and industrially scalable way.

Hence, an object of the present invention is a method for the prepation of an intermediate of formula 1i

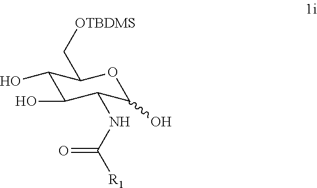

wherein R₁ is a saturated C₅-C₁₅ alkyl chain, comprising the following steps
1) Selective acylation of the amino group in the C₂ position of glucosamine hydrochloride by reaction with acyl chloride in the presence of sodium bicarbonate. 2) Protection by selective silylation of hydroxyl in position C by reaction with tert-butyldimethylsilyl chloride (TBDMSCI) in the presence of imidazole.

Therefore, present invention also relates to an intermediate of formula 1i

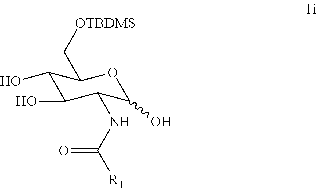

wherein R₁ is a saturated C₅-C₁₅ alkyl chain.

According to an embodiment of the invention, said R₁ is free from any substituent.

Further, the present invention relates to a method for the preparation of compounds of formula 1 as defined in any of the embodiments above and in the claims

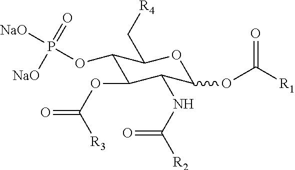

wherein R₁ is a saturated C₅-C₁₅ alkyl chain,

wherein R₂ is a saturated C₅-C₁₅ alkyl chain,

wherein R₃ is a saturated C₅-C₁₅ alkyl chain,

wherein R₄ is any substituent known by the skilled person that can be linked by means of a bond between C₆ and a suitable atom and/or any substituent which possesses an oxygen or a nitrogen atom that can bind to C comprising the following steps:
1) Selective acylation of the amino group in the C₂ position of glucosamine hydrochloride by reaction with acyl chloride in the presence of sodium bicarbonate.
2) Protection by selective silylation of hydroxyl in position C by reaction with tert-butyldimethylsilyl chloride (TBDMSCI) in the presence of imidazole obtaining the intermediate of formula 1i as defined in the previous described embodiments.
3) Selective acylation of hydroxyls in positions C₁ and C₃ by reaction with acyl chloride in the presence of triethylamine and N, N-dimethyl aminopyridine (DMAP).
4) Phosphorylation of hydroxyl in the C₄ position by reaction with dibenzyl N, N-diisopropylphospharamidite in the presence of triflate imidazolium, followed by oxidation of phosphite to phosphate via metachloroperbenzoic acid.
5) Deprotection of hydroxyl from silane in position C through the presence of sulfuric acid in catalytic quantities.
6) Deprotection of phosphate from benzyls in position C₄ and optionally deprotection of benzyls on any substituent in position C through hydrogenation catalyzed by Palladium on Carbon (Pd/C).

The method can alternatively start from the intermediate of formula 1i as defined above and in the claims and may comprise steps 3-6.

In one embodiment, the method of synthesis describved above may comprise an additional step 5i) after step 5) and before step 6)

5i) Phosphorylation of hydroxyl in position C by reaction with dibenzyl N, N-diisopropylphospharamidite in the presence of triflate imidazolium, followed by oxidation of phosphite to phosphate via metachloroperbenzoic acid, and wherein the resulting R₄ is a phosphate group (PO₄²⁻).

When carried out, this method leads to compounds of formula 1 wherein R₄ is a phosphate group, such as compounds of formulas 7, 8 and 9.

Alternatively, the method of synthesis may further comprise a step 5ii) instead of step 5i) after step 5) and before step 6):

5ii) Acylation of hydroxyl in C position either by reaction with carboxylic acid in the presence of a suitable condensing agent and catalyst, such as 1-ethyl-3-(3-dimethylaminopropyl) carbodiimide (EDC) and N, N-dimethyl aminopyridine (DMAP), or by reaction with acyl chloride in the presence of a suitable catalyst, such as N, N-dimethyl aminopyridine (DMAP).

wherein the resulting R₄ is an acyl group. When carried out, this method leads to compounds of formula 1 wherein R₄ is an acyl group, such as compounds of formulas 10 and 11.

In a preferred embodiment, said suitable condensing agent and catalyst are 1-ethyl-3-(3-dimethylaminopropyl) carbodiimide (EDC) and N, N-dimethyl aminopyridine (DMAP).

Alternatively, the method of synthesis may further comprise a step 5iii) instead of step 5i) and 5ii) after step 5) and before step 6):

5iii) Glycosylation of hydroxyl in C position by reaction of a glycosyl chloride donor in the presence of silver (I) oxide as activator, of triflic acid as catalyst and molecular sieves as water scavenger

Or

Glycosylation of hydroxyl in C position by reaction of a glycosyl thioethyl (Set) donor in the presence of NIS (N-iodosuccinimide) as activator and HOFox (3,3-difluoroxindole) as catalyst and molecular sieves as water scavenger, wherein the resulting R₄ is a glycosyl group.

Alternatively, the method of synthesis may further comprise a step 5iv) instead of step 5i), 5ii) and 5iii) after step 5) and before step 6):

5iv) Alkylation of hydroxyl in C by reaction of a stabilized alkyl chloride in the presence of silver (I) oxide as activator, of triflic acid as catalyst and molecular sieves as water scavenger wherein the resulting R₄ is n alkyl group.

Alternatively, the method of synthesis may further comprise a step 5v) and a step 5vi) instead of step 5i), 5ii), 5iii) and 5iv) after step 5) and before step 6):

5v) Tosylation of position C by reaction of tosyl chloride in presence of triethylamine as base and of DMAP as catalyst

5vi) azide instertion in position C by reaction with sodium azide in the presence of tetrabutylammonium iodide

wherein the resulting R₄ is an azide group.

Alternatively, the method of synthesis may further comprise a step 5vii) and a step 5viii) instead of step 5i), 5ii), 5iii) and 5iv) after step 5) and before step 6):

5vii) Glycosylation of hydroxyl in C position by reaction of a glycosyl chloride donor bearing a picoloyl group in the presence of Bi(OTf)₃ as sole activator. wherein the resulting R₄ is a glycosyl group.

5viii) Picoloyl group removal by reaction with Cu(OAc)₂.

According to the present description, the acylations described in reactions 1), 3), 5ii) can be carried out according to methods commonly used by technicians in the chemical field. By way of example, acylations can be carried out using acyl chloride or carboxylic acid in presence other common condensing agents, such as 1-ethyl-3-(3-dimethylaminopropyl) carbodiimide (EDC) or dicyclohexylcarbodiimide (DCC).

The condensations referred to in reactions 1) and 3) can be carried out using alkyl chains of different lengths, between 5 and 15 carbon atoms, thereby obtaining different derivatives of the molecule described by formula 1.

The protection of C described by reaction 2) can be carried out in accordance with the most common techniques known to those skilled in the chemical field. One of said common techniques is silylation in the presence of various non nucleophilic bases, such as triethylamine, diisopropylethylamine or sodium bicarbonate and catalyst such as N, N-dimethyl aminopyridine (DMAP).

The phosphorylation of C₄ described by reaction 4) can be carried out according to the most common techniques known to those skilled in the chemical field, such as phosphite insertion in presence of different acidic pH buffers, such as, but not limited to, tetrazole or 4,5-Dicyanoimidazole. The subsequent oxidation can be carried out by reaction with different mild oxidants, such as but not limited to dimethyldioxirane (DMDO) or tert-Butyl peroxide (tBuOOH).

The deprotection of C described by reaction 5) can be carried out according to the most common techniques known to those skilled in the chemical field. such as desilylation in the presence of tetrabutylammonium fluoride (TBAF), acetic acid (AcOH) or various types of acidic resins, i.e. IRA 120 H⁺, IRC 120 H⁺ or Dowex® 50W.

The process of synthesis according to the invention enables to make in an easy and industrially scalable way the compounds of formula 1.

As stated above, the invention encompasses both a as well as p anomers of the compound of formula 1 as defined above. The inventors have surprisingly found that, depending on the temperature and amount of a suitable catalyst of the acylation step 3, α or β anomers can be obtained.

Therefore, when a β anomer is desired, the acylation step 3) is carried out at a temperature ranging from −78° C. to 0° C. and with an amount of DMAP ranging from 0.05 to 0.2 equivalents.

In a preferred embodiment, in order to synthesise a β anomer acylation step 3 is carried out at −20° C. with 0.1 equivalents of DMAP.

On the other hand, when an α anomer is desired, the acylation step 3) is carried out at a temperature ranging from 20° C. to 50° C. with an amount of DMAP ranging from 2 to 2.5 equivalents.

In a preferred embodiment, in order to synnthesise an α anomer acylation step 3 is carried out at a temperature of about 30° C. with 2.02 equivalents of DMAP.

The methods as defined herein allow the synthesis of each one of the embodiments of the compounds of formula 1 (such as compounds of fomulas 2-12) as disclosed in the present specification. The skilled person will know the substituents to use based on the common knowledge in organic chemistry.

Advantageously, the invention also provides a method for the synthesis of a compound of formula X

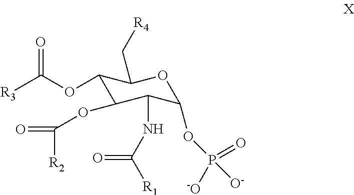

wherein R₁ is a saturated C₅-C₁₅ alkyl chain,

wherein R₂ is a saturated C₅-C₁₅ alkyl chain,

wherein R₃ is a saturated C₅-C₁₅ alkyl chain,

wherein R₄ is OH and wherein each of R₁, R₂ and R₃ is free from —OH substituents in position C₂

comprising the following steps:
1) Selective acylation of the amino group in the C₂ position of glucosamine hydrochloride by reaction with acyl chloride in the presence of sodium bicarbonate.
2) Protection by selective silylation of hydroxyl in position C by reaction with tert-butyldimethylsilyl chloride (TBDMSCI) in the presence of imidazole obtaining the intermediate of formula 1i as defined in the previous described embodiments.
3) Complete acylation of hydroxyls in positions C₁, C₃ and C₄ by reaction with acyl chloride in the presence of triethylamine and N, N-dimethyl aminopyridine (DMAP).
4) selective diacylation of position C₁ by reaction with ethylendiamine in presence of acetic acid
5) Phosphorylation of hydroxyl in the C₁ position by reaction with dibenzyl N, N-diisopropylphospharamidite in the presence of triflate imidazolium, followed by oxidation of phosphite to phosphate via metachloroperbenzoic acid.
6) Deprotection of hydroxyl from silane in position C through the presence of a 5% solution of sulfuric acid in water in catalytic quantities.
7) Deprotection of phosphate from benzyls in position C₄ and optionally deprotection of benzyls on any substituent in position C through hydrogenation catalyzed by Palladium on Carbon (Pd/C).

According to an embodiment of the invention, said R₁ R₂ and R₃ are free from any substituent.

The present invention also relates to the use of an intermediate compound of formula 1i, as defined in any one of the embodiments herein disclosed, for the synthesis of compounds of formula 1

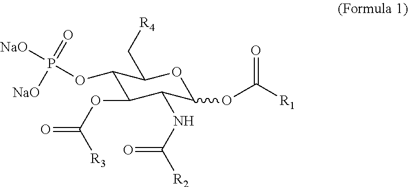

wherein R₁ is a saturated C₅-C₁₅ alkyl chain,

wherein R₂ is a saturated C₅-C₁₅ alkyl chain,

wherein R₃ is a saturated C₅-C₁₅ alkyl chain,

wherein R₄ is any substituent that can be linked by means of a bond between Cᵣ and a suitable atom and/or any substituent which possesses an oxygen or a nitrogen atom that can bind to C₆.

According to an embodiment of the invention, said R₁ R₂ and R₃ are free from any substituent.

Furthermore, the present invention relates to the use of an intermediate of formula 1i as defined in any one of the embodiments herein disclosed, for the synthesis of compounds of formula X

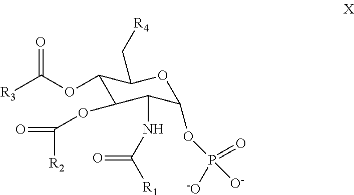

wherein R₁ is a saturated C₅-C₁₅ alkyl chain,

wherein R₂ is a saturated C₅-C₁₅ alkyl chain,

wherein R₃ is a saturated C₅-C₁₅ alkyl chain,

wherein R₄ is OH and wherein each of R₁, R₂ and R₃ is free from —OH substituents in position C₂.

According to an embodiment of the invention, said R₁ R₂ and R₃ are free from any substituent.

Object of the invention is also a process for the preparation of pharmaceutical formulations or of vaccine compositions comprising the steps of the above process, and at least one step wherein the product obtained at 6) in a pharmaceutically acceptable grade is mixed with at least one pharmaceutically acceptable carrier and/or excipient.

In any part of the present description and claims the term comprising can be substituted by the term “consisting of”.

In compliance with Art. 170bis of the Italian patent law it is herein declared that: all experiments involving cells were carried out on commercially available cells with reference to model mice used in the described experiments, the obligations deriving from the national or EU regulations, and in particular, from the provisions referred to in paragraph 6 of Legislative Decree No. 206 of 12 Apr. 2001 and 8 Jul. 2003 no. 224, have been fulfilled.

### EXAMPLES

### Chemistry

All reagents and solvents were purchased from commercial source and used without further purifications, unless stated otherwise. Reactions were monitored by thin-layer chromatography (TLC) performed over Silica Gel 60 F254 plates (Merck®). Flash chromatography purifications were performed on silica gel 60 60-75 μm from commercial source.

1H and 13C NMR spectrum were recorded with Bruker Advance 400 with TopSpin® software, or with NMR Varian 400 with Vnmrj software. Chemical shifts are expressed in ppm respect Me4Si; coupling constants are expressed in Hz. The multiplicity in the 13C spectra was deducted by APT experiments.

### Synthesis of 13

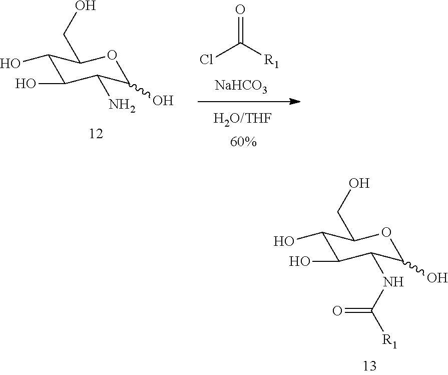

Glucosamine hydrochloride 12 (10 g, 46.5 mmol, 1 eq.) and NaHCO₃ (10.54 g, 126 mmol, 2.7 eq.) were dissolved in water (120 ml). Then, previously dissolved lauroyl chloride (11.20 g, 51.2 mmol, 1,1 eq.) in THF (120 ml) was added dropwise to the solution at 0° C. Reaction was stirred for 5 h, then solution was filtered. A white solid was obtained, which was washed with 4° C. water and THF. Excess water was then coevaporated with toluene under reduced pressure, to obtain the desired product 13 as a white powder in 60% yield (10.10 g). Compound was used without further purification.

1H NMR (400 MHz, DMSO) δ 7.68 (d, J=8.1 Hz, 1H), 7.52 (d, J=7.7 Hz, 3H), 6.46 (d, J=6.3 Hz, 1H), 6.37 (d, J=4.0 Hz, 3H), 4.97-4.86 (m, 7H), 4.81 (d, J=4.7 Hz, 1H), 4.62 (d, J=5.0 Hz, 3H), 4.53 (t, J=5.7 Hz, 1H), 4.43 (dd, J=9.5, 4.3 Hz, 4H), 3.73-3.42 (m, 18H), 3.34-3.22 (m, 2H), 3.16-3.09 (m, 3H), 3.04 (d, J=14.1 Hz, 2H), 2.13-2.03 (m, 8H), 1.56-1.37 (m, 9H), 1.26 (d, J=14.5 Hz, 67H), 0.86 (t, J=6.8 Hz, 13H).

13C NMR (101 MHz, DMSO) δ 173.31, 172.82, 96.11, 91.05, 77.21, 74.74, 72.49, 71.59, 71.32, 70.83, 61.58, 57.57, 54.73, 40.59, 40.38, 40.17, 39.96, 39.75, 39.54, 39.33, 36.18, 35.74, 31.77, 29.53, 29.49, 29.43, 29.37, 29.23, 29.18, 29.14, 25.78, 22.56, 14.42.

### Synthesis of 14

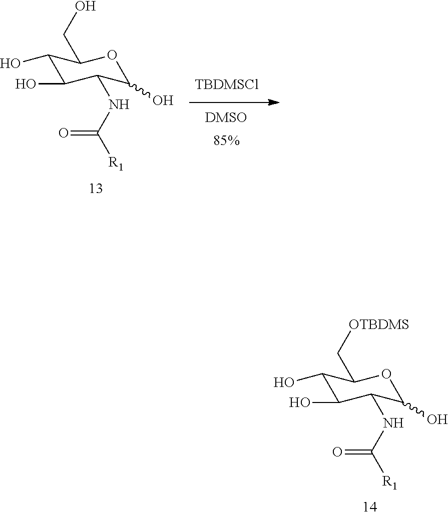

To a solution of 13 (3 g, 8.3 mmol, 1 eq.) and imidazole (850 mg, 12.4 mmol, 1.5 Eq) in dimethylsulfoxide (166 ml, 0.05 M) a solution of TBDMSCI (1.4 g, 9.1 mmol, 1.1 eq.) in DCM (15 ml) was added dropwise under inert atmosphere in ice bath. Subsequently, the solution was allowed to return at room temperature and stirred overnight. Reaction, monitored by TLC (DCM/MeOH 9:1), was then stopped and the solution concentrated under reduced pressure. Then it was diluted with AcOEt and washed three times with NH₄Cl. Organic phase thus obtained was dried with Na₂SO₄ and solvent was removed by rotavapor. Raw product thus obtained (3.65 g) was resuspended in EtPet at 0° C. for 30 min. Then, suspension was filtered under vacuum and desired compound was recovered as a white solid. After purification, 3.5 g of compound 14 as a whiteish solid were obtained, in 85% yield.

1H NMR (400 MHz, DMSO) δ 7.62 (d, J=7.9 Hz, 1H), 6.43 (d, J=6.4 Hz, 1H), 4.90 (t, J=6.5 Hz, 1H), 4.77 (t, J=9.1 Hz, 1H), 4.42 (t, J=7.0 Hz, 1H), 3.86 (d, J=10.8 Hz, 1H), 3.66 (dd, J=11.0, 4.6 Hz, 1H), 3.30 (d, J=7.9 Hz, 1H), 3.14-2.98 (m, 1H), 2.06 (t, J=7.4 Hz, 1H), 1.48 (s, 1H), 1.24 (s, 3H), 0.94-0.74 (m, 2H), 0.05 (d, J=3.0 Hz, 1H).

13C NMR (101 MHz, DMSO) δ 173.21, 95.93, 77.09, 74.83, 70.82, 63.61, 57.54, 40.61, 40.40, 40.20, 39.99, 39.78, 39.57, 39.36, 36.20, 31.78, 29.54, 29.51, 29.45, 29.39, 29.20, 29.14, 26.41, 25.76, 22.57, 18.64, 14.41, −4.66, −4.67.

### Synthesis of 15

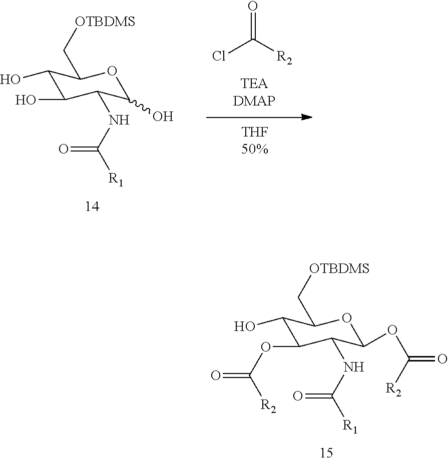

Compound 14 (2.0 g, 4.2 mmol, 1 eq.) and 4-dimethylaminopyridine (26 mg, 0.2 mmol, 0.05 Eq) were dissolved in anhydrous THF (84 ml, 0.05 M) under Ar atmosphere. Triethylamine (2.4 ml, 17.2 mmol, 4.1 Eq) and lauroyl chloride (2.10 ml, 8.5 mmol, 2.0 eq.) were added dropwise to the solution at −20° C. Reaction was stirred for two hours at −20° C., then controlled by TLC (EtPet/AcOEt 6:4). Subsequently, solution was diluted in AcOEt and washed with 1M HCl. Organic phase thus obtained was dried with Na₂SO₄ and solvent was removed by rotavapor. Raw product thus obtained (4 g) was purified using flash column chromatography (Tol/AcOEt 9:1). After purification, 2.1 g of compound 15 were obtained, in 50% yield.

1H NMR (400 MHz, DMSO) δ 7.80 (d, J=9.5 Hz, 1H), 5.56 (d, J=8.9 Hz, 1H), 5.38 (d, J=5.9 Hz, 1H), 4.92 (dd, J=10.6, 8.6 Hz, 1H), 3.83 (dd, J=10.4, 5.8 Hz, 2H), 3.76-3.70 (m, 1H), 3.38 (dd, J=14.3, 8.5 Hz, 2H), 2.30-2.14 (m, 7H), 1.94 (t, J=7.3 Hz, 2H), 1.44 (dd, J=25.9, 6.4 Hz, 10H), 1.24 (d, J=2.4 Hz, 75H), 0.90-0.81 (m, 24H), 0.07-0.01 (m, 6H).

13C NMR (101 MHz, DMSO) δ 174.93, 172.72, 172.34, 171.74, 92.49, 77.48, 75.66, 67.73, 62.54, 52.26, 40.65, 40.44, 40.23, 40.02, 39.82, 39.61, 39.40, 36.08, 34.13, 33.94, 31.78, 31.74, 29.59, 29.52, 29.50, 29.44, 29.39, 29.35, 29.30, 29.19, 29.00, 28.93, 28.75, 26.26, 25.70, 24.95, 24.77, 22.55, 18.54, 14.39, 14.36, −4.71, −4.78.

### Synthesis of 16

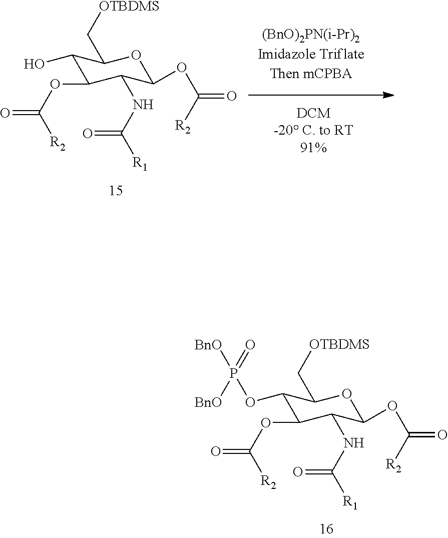

Compound 15 (2.12 g, 2.4 mmol, 1 eq.) and imidazole triflate (1.4 g, 5.4 mmol, 2.25 Eq) were dissolved in DCM (121 mL, 0.02 M) under inert atmosphere. Dibenzyl N,N-diisopropylphosphoramidite (1.83 g, 5.3 mmol, 2.2 eq) was added to the solution at 0° C. Reaction was monitored by TLC (EtPet/acetone 9:1); after 30 min, substrate depletion was detected. Solution was then cooled at −20° C. and meta-chloroperbenzoic acid (1.66 g, 9.7 mmol, 4 Eq), dissolved in 17 ml of DCM, was added dropwise. After 30 min the reaction was allowed to return to RT and left stirring overnight.

After TLC analysis, reaction was quenched with 15 ml of a saturated NaHCO₃ solution and concentrated by rotavapor. The mixture was then diluted in AcOEt and washed 3 times with a saturated NaHCO₃ solution and three times with a 1 M HCl solution. The organic phase was recovered, dried with Na₂SO₄ and solvent was removed by rotavapor. Crude thus obtained was purified by flash column chromatography (EtPet/acetone 9:1). 2.41 g of pure compound 16 were obtained as a yellow oil in a 91% yield.

1H NMR (400 MHz, CDC3) δ 7.34-7.25 (m, 10H), 5.61 (d, J=8.7 Hz, 1H), 5.44 (d, J=9.6 Hz, 1H), 5.16 (dd, J=10.8, 9.1 Hz, 1H), 5.00 (dd, J=8.1, 2.8 Hz, 2H), 4.96-4.91 (m, 2H), 4.53 (q, J=9.2 Hz, 1H), 4.23 (dt, J=10.8, 9.5 Hz, 1H), 3.91 (dd, J=11.9, 1.8 Hz, 1H), 3.78 (dd, J=11.9, 4.6 Hz, 1H), 3.56 (ddd, J=9.6, 4.4, 1.7 Hz, 1H), 2.31 (td, J=7.5, 3.5 Hz, 2H), 2.19 (t, J=7.7 Hz, 2H), 2.07-2.01 (m, 2H), 1.61-1.37 (m, 6H), 1.33-1.10 (m, 50H), 0.92-0.83 (m, 19H), 0.03-0.03 (m, 6H).

13C NMR (101 MHz, CDCl3) δ 174.43, 172.75, 172.40, 135.52, 128.60, 128.56, 127.88, 127.83, 92.59, 77.31, 77.00, 76.68, 76.23, 76.16, 72.94, 72.89, 69.56, 69.51, 69.46, 61.63, 52.79, 36.76, 34.08, 33.94, 31.89, 29.65, 29.60, 29.49, 29.47, 29.43, 29.37, 29.33, 29.25, 29.11, 29.01, 25.82, 25.58, 24.63, 24.58, 22.66, 18.32, 14.07, −5.19, −5.32.

### Synthesis of 17

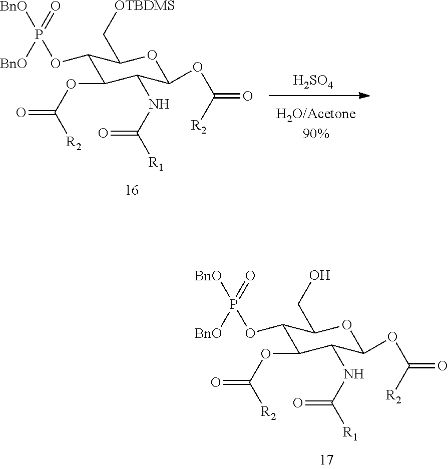

Compound 16 (2.41 g, 2.4 mmol, 1 Eq) was dissolved in acetone (48 mL) and a 5% v/v solution of H₂SO₄ in H₂O was added at RT (480 μL, 1% v/v). Solution was left stirring for 8 h and monitored by TLC (EtPet/Acetone 8:2). After reaction completion, solution was diluted in AcOEt and washed three times with a saturated NaHCO₃ solution. Organic phase thus obtained was dried with Na₂SO₄ and solvent was removed by rotavapor. Raw product thus obtained was purified by flash column chromatography (EtPet/Acetone 85:15). After purification (2.1 g) of compound 17 was obtained as a white solid in a 90% yield.

1H NMR (400 MHz, CDCl3) δ 7.40-7.27 (m, 1H), 5.63 (d, J=8.8 Hz, 1H), 5.45 (d, J=9.6 Hz, 1H), 5.18 (dd, J=10.7, 9.3 Hz, 1H), 5.08-4.91 (m, 1H), 4.54 (q, J=9.5 Hz, 1H), 4.26 (dd, J=19.9, 9.3 Hz, 1H), 3.87-3.74 (m, 1H), 3.47 (d, J=9.7 Hz, 1H), 2.40-2.24 (m, 1H), 2.10-1.91 (m, 1H), 1.61-1.46 (m, 1H), 1.46-1.33 (m, 1H), 1.33-1.01 (m, 5H), 0.92-0.83 (m, 1H).

13C NMR (101 MHz, CDCl3) δ 174.11, 172.77, 172.49, 128.94, 128.84, 128.72, 128.66, 128.26, 127.95, 92.61, 77.33, 77.01, 76.69, 75.90, 75.87, 72.46, 72.42, 72.15, 72.10, 70.23, 70.17, 70.10, 60.23, 52.78, 36.71, 34.03, 33.71, 31.90, 29.67, 29.62, 29.49, 29.44, 29.38, 29.34, 29.32, 29.26, 29.23, 29.04, 29.01, 25.56, 24.59, 24.48, 22.66, 14.09.

### Synthesis of 1

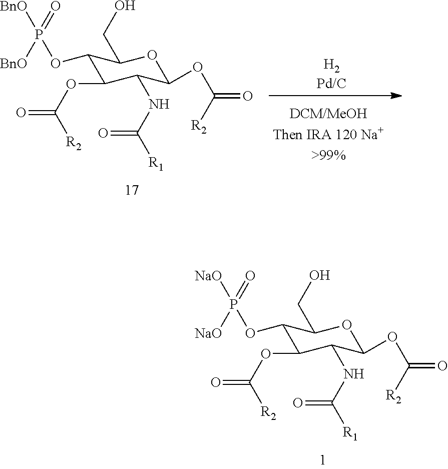

Compound 17 (50 mg, 0.05 mmol, 1 Eq) was dissolved in a mixture of DCM (2.5 mL) and MeOH (2.5 mL) and put under Ar atmosphere. Pd/C catalyser (10 mg, 20% m/m) was then added to the solution. Gases were then removed in reaction environment, which was subsequently put under H₂ atmosphere. The solution was allowed to stir for 2 h, then H₂ was removed and reaction monitored by TLC (EtPet/acetone 8:2). Triethylamine (100 μL) was then added to reaction, which was stirred for 15 min. Solution was subsequently filtered on syringe filters PALL 4549T Acrodisc 25 mm with GF/0.45 μm Nylon to remove Pd/C catalyser and solvents were evaporated by rotavapor. Crude product was resuspended in a DCM/MeOH solution and IRA 120 H⁺ was added. After 30 min stirring, IRA 120 H⁺ was filtered, solvents were removed by rotavapor, the crude resuspended in DCM/MeOH and IRA 120 Na⁺ was added. After 30 min stirring, IRA 120 Na⁺ was filtered and solvents were removed by rotavapor. (45 mg) of 1 were obtained as a white powder in a quantitative yield.

1H NMR (400 MHz, cd3od) δ 5.75 (d, J=8.9 Hz, 1H), 5.28 (t, J=9.8 Hz, 1H), 4.28 (q, J=9.7 Hz, 1H), 4.06 (t, J=9.6 Hz, 1H), 3.89-3.74 (m, 2H), 3.62 (t, J=9.2 Hz, 1H), 2.42-2.25 (m, 5H), 2.09 (t, J=7.6 Hz, 2H), 1.56 (d, J=6.4 Hz, 7H), 1.29 (s, 53H), 0.90 (t, J=6.6 Hz, 9H).

13C NMR (101 MHz, MeOD) δ 174.69, 173.32, 172.00, 92.16, 76.22, 76.17, 72.81, 72.78, 72.20, 72.14, 60.30, 52.82, 48.23, 48.02, 47.81, 47.59, 47.38, 47.17, 46.96, 36.05, 33.64, 33.55, 31.67, 31.66, 29.45, 29.39, 29.38, 29.35, 29.26, 29.20, 29.19, 29.14, 29.07, 29.05, 29.02, 28.92, 28.74, 25.58, 24.38, 22.31, 13.00.

### Synthesis of 18

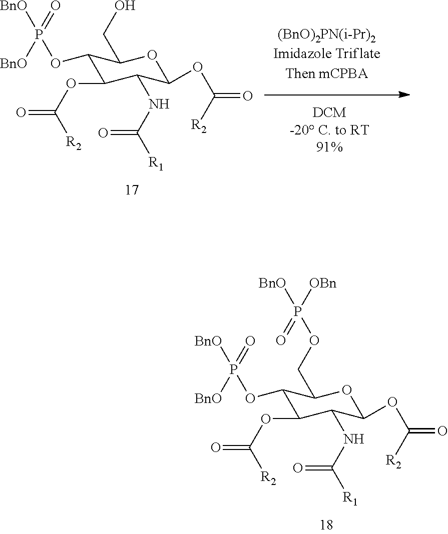

Compound 17 (2.36 g, 2.4 mmol, 1 eq.) and imidazole triflate (1.4 g, 5.4 mmol, 2.25 Eq) were dissolved in DCM (121 mL, 0.02 M) under inert atmosphere. Dibenzyl N,N-diisopropylphosphoramidite (1.83 g, 5.3 mmol, 2.2 eq) was added to the solution at 0° C. Reaction was monitored by TLC (EtPet/acetone 9:1); after 30 min, substrate depletion was detected. Solution was then cooled at −20° C. and meta-chloroperbenzoic acid (1.66 g, 9.7 mmol, 4 Eq), dissolved in 17 ml of DCM, was added dropwise. After 30 min the reaction was allowed to return to RT and left stirring overnight.

After TLC analysis, reaction was quenched with 15 ml of a saturated NaHCO₃ solution and concentrated by rotavapor. The mixture was then diluted in AcOEt and washed 3 times with a saturated NaHCO₃ solution and three times with a 1 M HCl solution. The organic phase was recovered, dried with Na₂SO₄ and solvent was removed by rotavapor. Crude thus obtained was purified by flash column chromatography (EtPet/acetone 9:1). 2.41 g of pure compound 18 were obtained as a yellow oil in a 91% yield.

1H NMR (400 MHz, CDCl3) δ 7.33-7.18 (m, 21H), 5.66 (d, J=8.8 Hz, 1H), 5.51 (d, J=9.5 Hz, 1H), 5.18 (dd, J=10.6, 9.2 Hz, 1H), 5.02 (dd, J=10.8, 3.3 Hz, 4H), 5.00-4.95 (m, 2H), 4.94-4.88 (m, 2H), 4.49-4.43 (m, 1H), 4.42-4.36 (m, 1H), 4.25 (dd, J=19.8, 9.3 Hz, 1H), 4.16 (ddd, J=11.8, 7.1, 5.0 Hz, 1H), 3.74 (dd, J=9.5, 4.2 Hz, 1H), 2.19 (dt, J=15.9, 7.0 Hz, 5H), 2.07-2.01 (m, 2H), 1.49 (dt, J=14.0, 7.1 Hz, 4H), 1.45-1.36 (m, 2H), 1.34-1.11 (m, 54H), 0.88 (t, J=6.8 Hz, 10H).

13C NMR (101 MHz, CDCl3) δ 174.22, 172.82, 172.18, 135.79, 135.72, 135.33, 128.62, 128.58, 128.52, 128.05, 128.00, 127.96, 92.44, 74.11, 72.59, 72.39, 69.38, 65.25, 52.68.

### Synthesis of 6

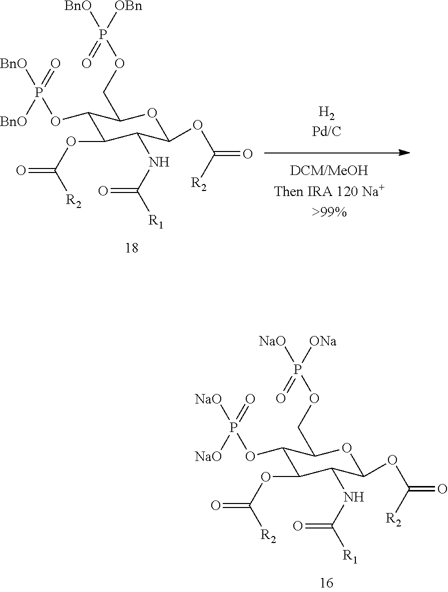

Compound 18 (57 mg, 0.05 mmol, 1 Eq) was dissolved in a mixture of DCM (2.5 mL) and MeOH (2.5 mL) and put under Ar atmosphere. Pd/C catalyser (10 mg, 20% m/m) was then added to the solution. Gases were then removed in reaction environment, which was subsequently put under H₂ atmosphere. The solution was allowed to stir for 2 h; then H₂ was removed and reaction monitored by TLC (EtPet/acetone 8:2).

Triethylamine (100 μL) was then added to reaction, which was stirred for 15 min. Solution was subsequently filtered on syringe filters PALL 4549T Acrodisc 25 mm with GF/0.45 μm Nylon to remove Pd/C catalyser and solvents were evaporated by rotavapor. Crude product was resuspended in a DCM/MeOH solution and IRA 120 H⁺ was added. After 30 min stirring, IRA 120 H⁺ was filtered, solvents were removed by rotavapor, the crude resuspended in DCM/MeOH and IRA 120 Na⁺ was added. After 30 min stirring, IRA 120 Na⁺ was filtered and solvents were removed by rotavapor. (45 mg) of 6 were obtained as a white powder in a quantitative yield.

1H NMR (400 MHz, cd3od) δ 5.77 (d, J=8.8 Hz, 1H), 5.32-5.23 (m, 1H), 4.39 (dd, J=18.9, 9.5 Hz, 1H), 4.21 (d, J=9.7 Hz, 3H), 4.10-4.00 (m, 1H), 3.80 (d, J=9.2 Hz, 1H), 2.44-2.24 (m, 6H), 2.09 (t, J=7.6 Hz, 2H), 1.55 (dd, J=13.5, 6.9 Hz, 10H), 1.39-1.24 (m, 79H), 0.96-0.82 (m, 33H).

13C NMR (101 MHz, MeOD) δ 174.87, 173.81, 91.22, 72.86, 72.80, 71.25, 68.94, 68.86, 68.80, 64.65, 64.61, 52.10, 48.24, 48.03, 47.82, 47.61, 47.39, 47.18, 46.97, 36.10, 35.63, 33.76, 33.64, 33.55, 33.40, 31.69, 31.63, 29.26, 29.22, 29.18, 29.15, 29.11, 29.06, 29.03, 28.98, 28.84, 28.78, 25.68, 25.62, 24.69, 24.63, 24.38, 22.35, 22.32, 13.06, 13.03, 7.82.

### Synthesis of 19

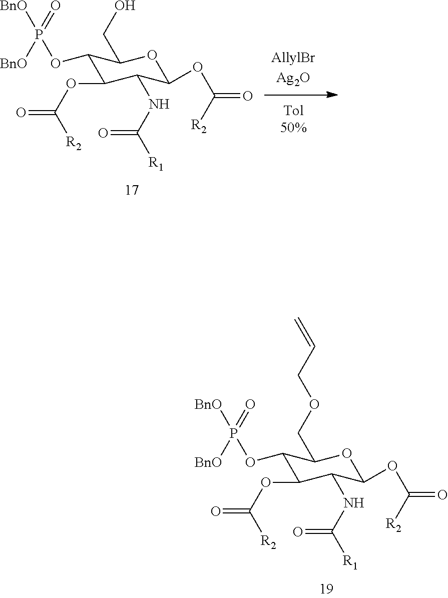

Compound 17 (100 mg, 0.1 mmol, 1 eq.) and silver (1) oxide (140 mg, 0.6 mmol, 6 Eq) were dissolved in toluene (1 mL, 0.1 M) under inert atmosphere. Allyl bromide (51 μL, 0.6 mmol, 6 eq) was added to the solution at RT. Reaction was left stirring overnight. After TLC analysis (EtPet/acetone 8:2), reaction was halted and solution filtered on a celite pad. The organic liquid phase was recovered and solvent was removed by rotavapor.

Crude thus obtained was purified by flash column chromatography (EtPet/acetone 8:2). 50 mg of pure compound 19 were obtained as a yellow oil in a 50% yield.

1 H NMR (400 MHz, CDCl3) δ 7.38-7.22 (m, 10H), 5.91-5.77 (m, 1H), 5.64 (d, J=8.8 Hz, 1H), 5.25-5.07 (m, 3H), 5.04-4.91 (m, 4H), 4.56 (q, J=9.3 Hz, 1H), 4.26 (dd, J=19.8, 9.3 Hz, 1H), 3.99-3.90 (m, 2H), 3.73 (dd, J=11.0, 1.5 Hz, 1H), 3.69 (dd, J=9.6, 4.4 Hz, 1H), 3.60 (dd, J=11.0, 4.4 Hz, 1H), 2.40-2.24 (m, 2H), 2.18 (dd, J=16.1, 8.4 Hz, 2H), 2.03 (dd, J=15.1, 7.1 Hz, 2H), 1.62-1.53 (m, 2H), 1.49 (dd, J=14.2, 7.2 Hz, 2H), 1.41 (dt, J=13.2, 6.8 Hz, 2H), 1.34-1.10 (m, 51H), 0.88 (t, J=6.8 Hz, 9H).

13C NMR (101 MHz, CDCl3) δ 174.33, 172.77, 172.41, 135.48, 134.43, 128.64, 128.59, 127.92, 117.20, 92.70, 77.32, 77.00, 76.68, 75.29, 75.23, 73.21, 73.15, 72.83, 72.47, 69.67, 69.63, 67.81, 52.83, 36.74, 34.05, 33.93, 31.89, 29.65, 29.62, 29.60, 29.49, 29.44, 29.36, 29.33, 29.31, 29.27, 29.23, 29.10, 29.00, 25.55, 24.57, 24.50, 22.65, 14.07.

### Synthesis of 21

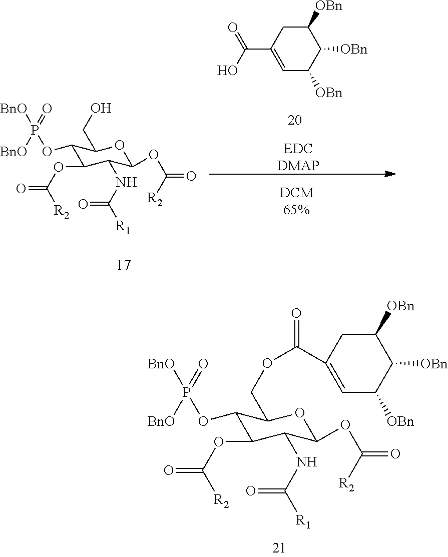

Compound 17 (100 mg, 0.1 mmol, 1 eq.) and compound 20 (48 mg, 0.11 mmol, 1.1 Eq) were dissolved in DCM (1 mL, 0.1 M) under inert atmosphere. 1-Ethyl-3-(3-dimethylaminopropyl) carbodiimide (EDC) (192 mg, 0.12 mmol, 1.2 eq) and dimethyl N,N aminopiridine (DMAP) was added to the solution at 0° C. Reaction was allowed to return to RT and left stirring overnight.

After reaction completion, solution was diluted in AcOEt and washed three times with a saturated NaHCO₃ solution. Organic phase thus obtained was dried with Na₂SO₄ and solvent was removed by rotavapor.

Crude thus obtained was purified by flash column chromatography (EtPet/acetone 85:15). 100 mg of pure compound 21 were obtained as a white powder in a 65% yield.

### Synthesis of 23

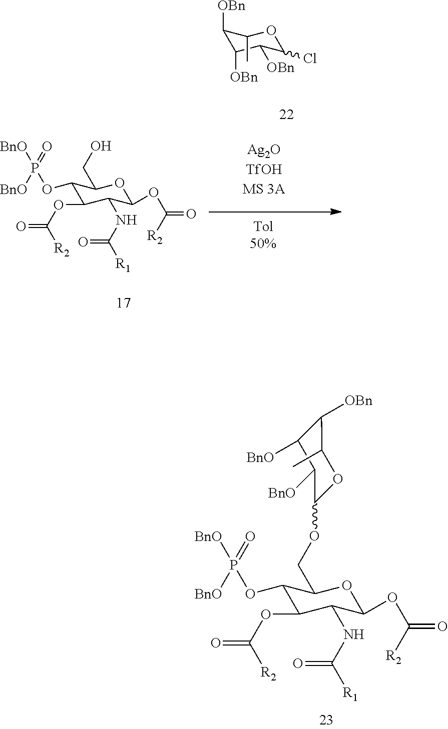

Compound 17 (100 mg, 0.1 mmol, 1 eq.), compound 22 (57 mg, 0.13 mmol, 1.25 eq.) and powdered 3 a molecular sieves (50 mg) were dissolved in toluene (1 mL, 0.1 M) under inert atmosphere and allowed to stir for 1 h. Silver (1) oxide (46 mg, 0.2 mmol, 2 Eq) was then added to the solution at RT, which was then cooled to 0° C. and triflic acid (4,4 μL, 0.05 mmol, 0.5 eq.) was added. Reaction was then left stirring overnight at RT. After TLC analysis (EtPet/acetone 8:2), reaction was halted and solution filtered on a celite pad. The organic liquid phase was recovered, diluted in AcOEt and washed three times with NaHCO₃. Organic phase was recovered, dried over Na₂SO₄ and evaporated. Crude thus obtained was purified by flash column chromatography (toluene/acetone 85:15). 50 mg of a mixture of diastereoisomers of compound 23 were obtained as a yellow oil in a 40% yield.

1H NMR (400 MHz, CDCl3) δ 7.40-7.20 (m, 38H), 5.65 (d, J=8.8 Hz, 1H), 5.61 (s, 1H), 5.40 (s, 1H), 5.24-5.18 (m, 1H), 5.14 (s, 1H), 4.91 (d, J=11.6 Hz, 9H), 4.69 (s, 2H), 4.62 (d, J=2.2 Hz, 4H), 4.57-4.37 (m, 2H), 4.23 (s, 1H), 2.40-2.26 (m, 3H), 2.13 (ddd, J=13.7, 7.6, 4.4 Hz, 3H), 2.05 (s, 3H), 1.57 (s, 6H), 1.47-1.36 (m, 3H), 1.29 (s, 79H), 0.88 (t, J=6.8 Hz, 14H).

13C NMR (101 MHz, CDCl3) δ 174.23, 172.81, 172.43, 138.66, 138.63, 135.24, 128.75, 128.69, 128.67, 128.62, 128.50, 128.40, 128.31, 128.27, 128.20, 128.06, 127.92, 127.88, 127.71, 127.64, 127.56, 127.41, 127.39, 98.55, 92.54, 80.36, 79.72, 77.32, 77.00, 76.68, 75.36, 74.99, 72.76, 72.51, 71.93, 69.72, 69.66, 69.63, 69.58, 68.09, 64.99, 52.74, 36.77, 34.03, 33.89, 31.89, 30.90, 29.66, 29.60, 29.49, 29.46, 29.36, 29.34, 29.22, 29.09, 29.02, 25.59, 24.58, 24.46, 22.66, 17.97, 14.09.

### Synthesis of 24

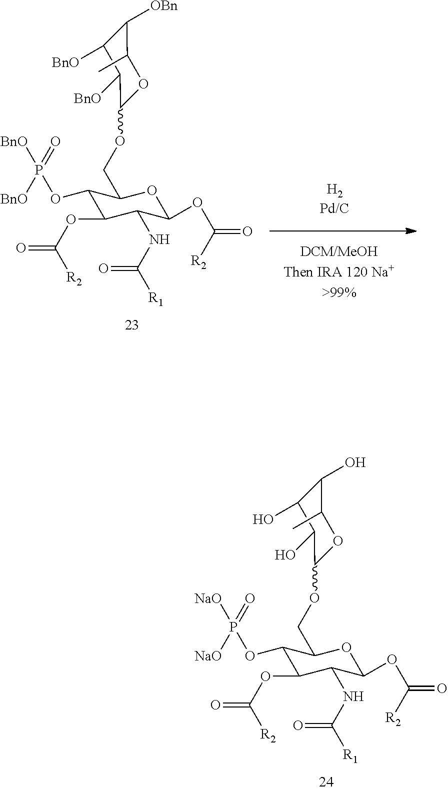

Compound 23 (70 mg, 0.05 mmol, 1 Eq) was dissolved in a mixture of DCM (2.5 mL) and MeOH (2.5 mL) and put under Ar atmosphere. Pd/C catalyser (10 mg, 20% m/m) was then added to the solution. Gases were then removed in reaction environment, which was subsequently put under H₂ atmosphere. The solution was allowed to stir for 2 h, then H₂ was removed and reaction monitored by TLC (EtPet/acetone 8:2). Triethylamine (100 μL) was then added to reaction, which was stirred for 15 min. Solution was subsequently filtered on syringe filters PALL 4549T Acrodisc 25 mm with GF/0.45 μm Nylon to remove Pd/C catalyser and solvents were evaporated by rotavapor. Crude product was resuspended in a DCM/MeOH solution and IRA 120 H⁺ was added. After 30 min stirring, IRA 120 H⁺ was filtered, solvents were removed by rotavapor, the crude resuspended in DCM/MeOH and IRA 120 Na⁺ was added. After 30 min stirring, IRA 120 Na⁺ was filtered and solvents were removed by rotavapor. (50 mg) of 24 were obtained as a white powder in a quantitative yield, as a mixture of diastereoisomers.

1H NMR (400 MHz, MeOD) δ 5.76 (dd, J=8.8, 4.7 Hz, 1H), 5.29 (dd, J=10.5, 9.1 Hz, 1H), 4.76 (d, J=1.2 Hz, 1H), 4.36 (dd, J=18.8, 9.4 Hz, 1H), 4.08 (ddt, J=19.6, 14.2, 5.9 Hz, 4H), 3.91 (dd, J=3.4, 1.6 Hz, 2H), 3.83-3.75 (m, 2H), 3.75-3.62 (m, 5H), 3.44-3.34 (m, 3H), 2.48-2.27 (m, 7H), 2.14-2.08 (m, 2H), 1.60 (s, 11H), 1.40-1.21 (m, 96H), 0.92 (t, J=6.8 Hz, 17H).

13C NMR (101 MHz, MeOD) δ 174.68, 173.39, 171.99, 101.03, 92.10, 75.14, 72.96, 72.66, 72.32, 70.90, 70.57, 68.47, 65.71, 52.71, 48.23, 48.02, 47.81, 47.59, 47.38, 47.17, 46.96, 36.06, 33.66, 33.57, 31.69, 29.39, 29.30, 29.09, 28.95, 28.76, 28.44, 25.59, 24.42, 22.33, 16.62, 13.03.

### Synthesis of 29

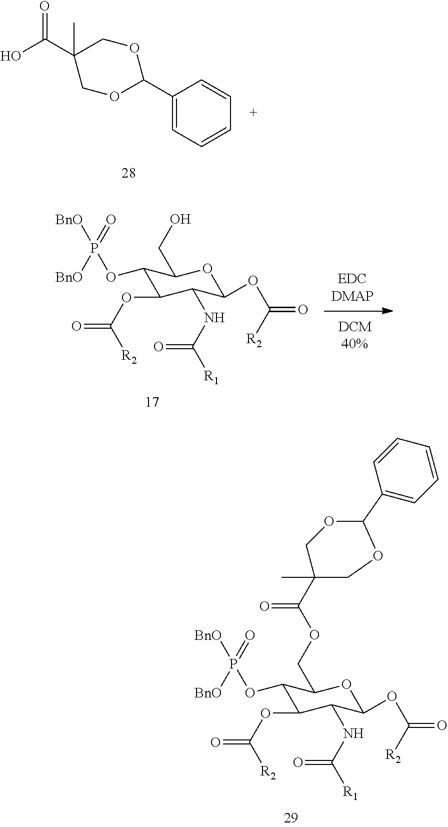

Compound 17 (100 mg, 0.11 mmol, 1 eq.) and compound 28 (24 mg, 0.11 mmol, 1.1 eq.) were dissolved in dry DCM (1 ml, 0.1 M) under Ar atmosphere. Then, EDC (23 mg, 0.12 mmol, 1.2 eq.) and DMAP (0.112 mg, 0.01 mmol, 0.1 eq.) were added to the solution at 0° C. Subsequently, the solution was allowed to return at room temperature and was stirred overnight. Reaction, monitored by TLC (EtPet/Acetone 8:2), was then stopped and the solution concentrated under reduced pressure. Then it was diluted with AcOEt and washed three times with HCl. Organic phase thus obtained was dried with Na2SO4 and solvent was removed by rotavapor. Raw product thus obtained (550 mg) was purified using flash column chromatography (EtPet/Acetone 85:15). After purification, 17 mg of compound 29 were obtained, in 40% yield.

1H NMR (400 MHz, CDCl3) δ 7.43 (dd, J=6.8, 2.9 Hz, 1H), 7.36-7.19 (m, 10H), 5.60 (d, J=8.7 Hz, 1H), 5.45 (s, 1H), 5.36 (d, J=9.7 Hz, 1H), 5.15 (dd, J=10.7, 9.1 Hz, 1H), 4.97 (t, J=9.9 Hz, 1H), 4.93-4.80 (m, 1H), 4.67 (ddd, J=18.4, 12.5, 2.3 Hz, 2H), 4.51 (q, J=9.3 Hz, 1H), 4.22 (td, J=12.5, 7.2 Hz, 1H), 3.79 (dd, J=9.5, 3.1 Hz, 1H), 3.64 (d, J=10.2 Hz, 1H), 2.36-2.23 (m, 1H), 2.21-2.09 (m, 1H), 2.09-1.99 (m, 1H), 1.04 (s, 2H), 0.98-0.75 (m, 5H).

13C NMR (101 MHz, CDCl3) δ 172.86, 134.44, 129.73, 128.98, 128.68, 128.27, 128.08, 127.97, 127.89, 126.22, 126.09, 101.61, 92.55, 77.31, 76.99, 76.67, 73.09, 72.47, 72.27, 69.82, 67.34, 61.47, 52.67, 50.48, 42.54, 36.74, 34.02, 31.89, 29.60, 29.44, 29.33, 28.99, 25.57, 24.50, 22.66, 19.14, 17.39, 14.08.

### Synthesis of 30

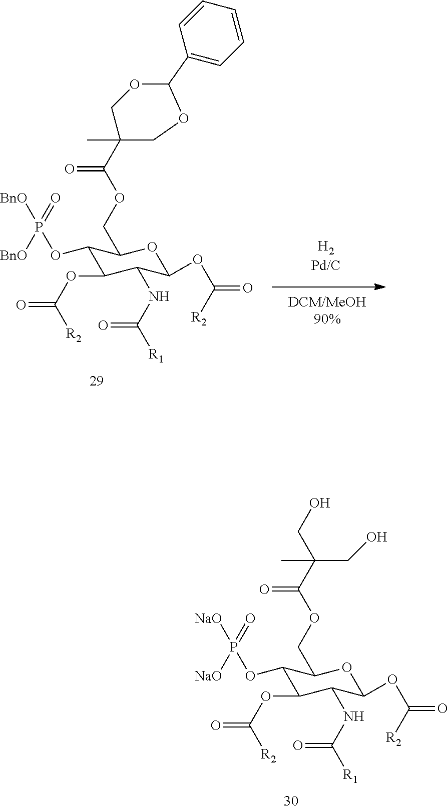

Compound 29 (30 mg, 0.03 mmol, 1 eq.) was dissolved in a 1:1 mixture of dry 0CM and dry MeOH (3 ml, 0.01 M) under Ar atmosphere. Then, Pd/C catalyst (6 mg), 20% i/i) was added always under inert atmosphere. The reaction is left under vacuum for some minutes. Then, the hydrogen was added, and the reaction stirred overnight at room temperature. Reaction, monitored by TLC (Toluene/Acetone 85:15), was then stopped. The hydrogen was completely removed and the Ar atmosphere restored. Triethylamine (60 □l) was added to the solution that stirred for 30 minutes. Then, the catalyst was removed removed by filtration on syringe filters PALL 4549T Acrodisc 25 mm with GF/0.45 μm Nylon to remove Pd/C catalyser and solvents were evaporated by rotavapor. Raw compound was resuspende in DCM/MeoH 1:1 and IRA 120 H+ was added to the solution. After 30 minutes, the acid resin was removed, and sodic resin was added instead. The solution was stirred again for 30 minutes; then Solvent was removed by rotavapor, obtaining 52.9 mg of compound 30 were obtained, in >99% yield.

¹H NMR (400 MHz, MeOD) δ 5.76 (d, J=8.9 Hz, 1H), 5.33-5.24 (m, 1H), 4.54-4.38 (m, 2H), 4.37-4.29 (m, 1H), 4.14-4.05 (m, 1H), 3.89 (d, J=7.3 Hz, 1H), 3.78 (q, J=9.0 Hz, 1H), 3.67 (dd, J=16.4, 11.6 Hz, 4H), 3.56 (t, J=6.6 Hz, 1H), 2.49-2.27 (m, 5H), 2.23-2.08 (m, 2H), 1.59 (d, J=6.4 Hz, 7H), 1.22-1.15 (m, 3H).

### Synthesis of 32

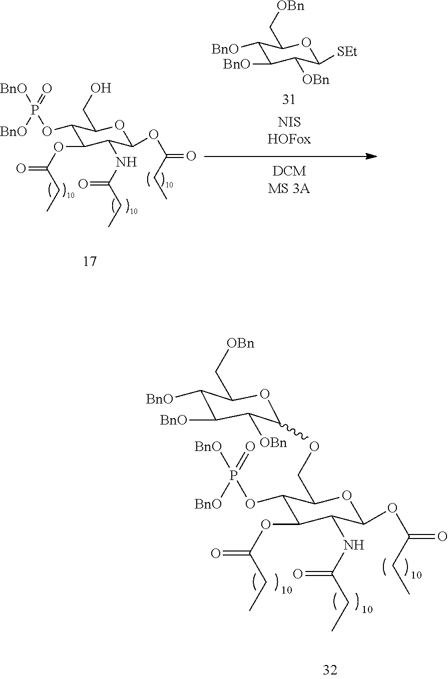

Compound 17 (100 mg, 0.1 mmol, 1 eq.), compound 31 (110 mg, 0.2 mmol, 2 eq.) and powdered 3a molecular sieves (330 mg) were dissolved in DCM (2 mL, 0.2 M) under inert atmosphere and allowed to stir for 1 h. NIS (45 mg, 0.2 mmol, 2 Eq) and HOFox (8.5 mg, 0.5 mmol, 0.5 eq.) were then added to the solution at RT. Reaction was then left stirring c.a 1.5 h at RT.

After TLC analysis (EtPet/acetone 8:2), reaction was stopped and solution filtered on a cotton pad. The organic liquid phase was recovered, diluted in AcOEt and washed three times with Na₂S2O₃. Organic phase was recovered, dried over Na₂SO₄ and evaporated. Crude thus obtained was purified by flash column chromatography (EtPet/acetone 80:20). 120 mg of a mixture of diastereoisomers of compound 32 were obtained as a yellow oil in a 84% yield.

1H NMR (400 MHz, CDCl3) δ 7.40-7.20 (m, 32H), 7.11 (ddd, J=13.3, 6.8, 2.7 Hz, 2H), 5.62 (dd, J=8.8, 3.8 Hz, 1H), 5.34 (d, J=9.5 Hz, 1H), 5.27 (d, J=9.6 Hz, OH), 5.13 (td, J=10.9, 8.9 Hz, 1H), 5.00-4.85 (m, 6H), 4.79 (dd, J=10.9, 2.5 Hz, 1H), 4.73 (d, J=11.0 Hz, 1H), 4.70-4.64 (m, 2H), 4.63-4.54 (m, 2H), 4.53-4.45 (m, 1H), 4.44-4.21 (m, 4H), 3.93 (t, J=9.3 Hz, 1H), 3.87-3.75 (m, 3H), 3.70-3.51 (m, 5H), 3.46-3.33 (m, 1H), 2.22 (t, J=7.6 Hz, 1H), 2.13 (dt, J=17.2, 7.7 Hz, 3H), 2.03 (q, J=7.3 Hz, 3H), 1.50 (s, 3H), 1.40 (p, J=7.1 Hz, 3H), 1.22 (d, J=16.1 Hz, 52H), 0.88 (t, J=6.7 Hz, 10H).

13C NMR (101 MHz, CDCl3) δ 174.35, 172.75, 172.22, 138.97, 138.22, 138.03, 128.74, 128.69, 128.67, 128.59, 128.45, 128.35, 128.29, 128.22, 128.12, 128.02, 127.91, 127.89, 127.83, 127.80, 127.73, 127.69, 127.63, 127.48, 127.43, 103.88, 97.27, 92.65, 92.54, 84.49, 81.90, 79.78, 77.61, 77.50, 77.34, 77.02, 76.70, 75.58, 74.95, 74.64, 73.43, 73.33, 73.27, 72.80, 72.71, 70.19, 69.73, 68.41, 52.92, 36.83, 34.03, 33.94, 31.93, 29.69, 29.64, 29.51, 29.47, 29.39, 29.37, 29.33, 29.26, 29.14, 29.04, 25.64, 24.60, 24.53, 24.36, 22.69, 14.12.

### Synthesys of 34

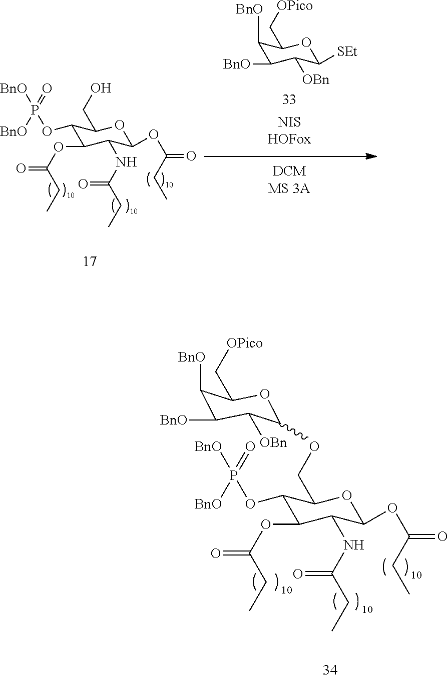

Compound 17 (100 mg, 0.1 mmol, 1 eq.), compound 33 (110 mg, 0.2 mmol, 2 eq.) and powdered 3a molecular sieves (330 mg) were dissolved in DCM (2 mL, 0.2 M) under inert atmosphere and allowed to stir for 1 h. Reaction was cooled at 0° C. and Bi(OTf)₃ (50 mg, 0.075 mmol, 0.75 Eq) and was then added to the solution. Reaction was then left stirring overnight at RT.

After TLC analysis (EtPet/acetone 7:3), reaction was stopped and solution filtered on a celite pad. The organic liquid phase was recovered, diluted in AcOEt and washed three times with NaHCO₃. Organic phase was recovered, dried over Na₂SO₄ and evaporated. Crude thus obtained was purified by flash column chromatography (EtPet/acetone 70:30), which allowed to separate the two diastereoisomers. 125 mg of of total compound 34 (α+β) were obtained as a yellow oil in a 94% yield.

34α

1 H NMR (400 MHz, CDCl3) δ 8.75 (dt, J=4.6, 1.4 Hz, 1H), 8.06 (d, J=7.8 Hz, 1H), 7.89 (td, J=7.7, 1.8 Hz, 1H), 7.47 (ddd, J=7.6, 4.7, 1.2 Hz, 1H), 7.40-7.13 (m, 30H), 5.53 (d, J=8.8 Hz, 1H), 5.32 (d, J=9.7 Hz, 1H), 5.10 (dd, J=10.8, 8.9 Hz, 1H), 4.99-4.90 (m, 5H), 4.89 (d, J=3.5 Hz, 1H), 4.83 (d, J=11.7 Hz, 1H), 4.75 (d, J=11.8 Hz, 1H), 4.68 (dd, J=11.7, 6.1 Hz, 2H), 4.61 (d, J=11.4 Hz, 1H), 4.40-4.30 (m, 2H), 4.30-4.25 (m, 1H), 4.16-4.09 (m, 1H), 4.05 (dd, J=10.2, 2.9 Hz, 2H), 3.98 (dd, J=10.1, 2.7 Hz, 1H), 3.90 (d, J=2.5 Hz, 1H), 3.78 (td, J=9.5, 5.4 Hz, 3H), 2.26 (q, J=7.4 Hz, 2H), 2.15 (d, J=7.8 Hz, 2H), 2.06-1.99 (m, 3H), 1.49 (q, J=7.4 Hz, 4H), 1.41 (p, J=7.3 Hz, 1H), 1.23 (d, J=7.7 Hz, 61H), 0.88 (qt, J=3.8, 1.8 Hz, 12H).

13C NMR (101 MHz, CDCl3) δ 174.24, 172.55, 172.30, 164.62, 149.93, 147.89, 138.46, 138.29, 137.14, 128.66, 128.39, 128.30, 128.07, 127.87, 127.69, 127.63, 127.46, 126.92, 125.40, 97.29, 92.61, 79.00, 76.34, 75.16, 74.57, 73.59, 73.19, 72.81, 69.70, 68.80, 65.18, 65.03, 52.82, 36.82, 33.97, 31.92, 29.64, 29.52, 29.36, 29.30, 29.14, 29.06, 25.63, 24.60, 22.69, 14.11.

34β

1H NMR (400 MHz, CDCl3) δ 8.73 (dd, J=4.9, 1.7 Hz, 1H), 7.95 (d, J=7.8 Hz, 1H), 7.78 (td, J=7.8, 1.8 Hz, 1H), 7.46-7.41 (m, 1H), 7.29 (ddddd, J=21.3, 16.3, 13.5, 8.4, 4.4 Hz, 27H), 5.63 (d, J=8.8 Hz, 1H), 5.36 (d, J=9.6 Hz, 1H), 5.12 (dd, J=10.8, 8.9 Hz, 1H), 4.97 (d, J=11.7 Hz, 1H), 4.95-4.86 (m, 5H), 4.81 (d, J=11.8 Hz, 1H), 4.71 (d, J=11.9 Hz, 1H), 4.65 (dd, J=11.2, 5.6 Hz, 2H), 4.39 (d, J=8.0 Hz, 1H), 4.48-4.34 (m, 2H), 4.34-4.22 (m, 2H), 3.90-3.82 (m, 3H), 3.68 (t, J=4.6 Hz, 1H), 3.64 (dd, J=9.2, 5.7 Hz, 1H), 3.51 (dd, J=9.8, 2.9 Hz, 1H), 2.19-2.06 (m, 3H), 2.02 (t, J=7.7 Hz, 2H), 1.49 (t, J=7.3 Hz, 2H), 1.40 (p, J=7.2 Hz, 1H), 1.35-1.04 (m, 55H), 0.88 (td, J=6.8, 2.1 Hz, 10H).

13C NMR (101 MHz, CDCl3) δ 174.24, 172.75, 172.16, 164.53, 149.91, 147.67, 138.78, 138.53, 138.20, 137.00, 128.65, 128.62, 128.51, 128.36, 128.28, 128.25, 128.04, 127.98, 127.62, 127.57, 127.51, 126.90, 125.35, 104.08, 92.54, 81.88, 79.16, 74.41, 73.25, 72.85, 72.11, 69.77, 68.46, 64.19, 52.87, 36.77, 33.89, 31.92, 29.69, 29.63, 29.36, 29.34, 29.22, 29.12, 28.99, 25.59, 24.60, 24.34, 22.69, 14.12.

### Synthesis of 35

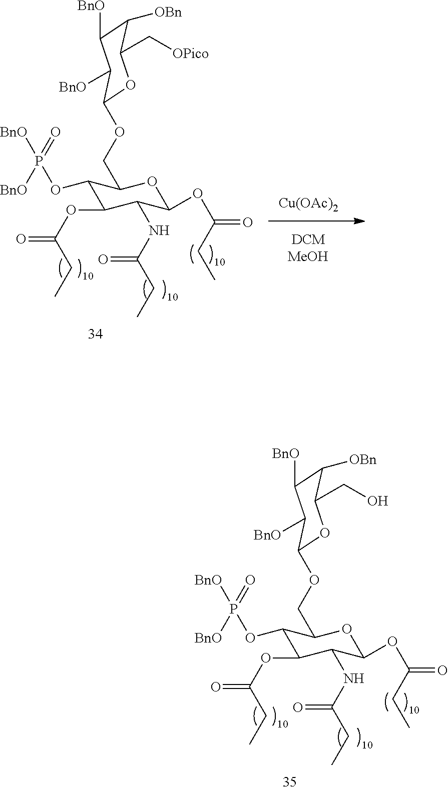

To a solution of 34 (75 mg, 0.5 mmol, 0.5 eq) in a 3:1 mixture of DCM/MeOH (5 mL, 0.1 M) under inert atmosphere, Cu(OAc)₂ (15 mg, 0.75 mmol, 1.5 eq.) was added at RT. Solution is left stirring for c.a. 2 h and then monitored by TLC (EtPet/AcOEt 6:4).

Solvent is evaporated by rotavapor and solution is purified by flash chromatography (EtPet/AcOEt 6:4) without further purification.

55 mg of compound 35 were recovere in a 80% yield

1H NMR (400 MHz, CDCl3) δ 7.40-7.22 (m, 29H), 5.62 (d, J=8.7 Hz, 1H), 5.38 (d, J=9.6 Hz, 1H), 5.12 (dd, J=10.8, 8.9 Hz, 1H), 4.97-4.88 (m, 6H), 4.80 (d, J=11.9 Hz, 1H), 4.71 (d, J=11.9 Hz, 1H), 4.65 (dd, J=13.1, 11.3 Hz, 2H), 4.39-4.36 (m, 1H), 4.35 (d, J=5.5 Hz, 1H), 4.33 (d, J=7.4 Hz, 1H), 4.27 (dt, J=10.8, 7.4 Hz, 1H), 3.86-3.80 (m, 2H), 3.72 (d, J=3.3 Hz, 2H), 3.72-3.63 (m, 2H), 3.47 (dd, J=9.7, 2.9 Hz, 1H), 3.39 (dd, J=11.5, 4.8 Hz, 1H), 3.31 (dd, J=7.2, 4.9 Hz, 1H), 2.16 (dd, J=8.8, 7.1 Hz, 2H), 2.09 (ddd, J=8.7, 7.2, 5.1 Hz, 2H), 2.03 (t, J=7.7 Hz, 2H), 1.51 (q, J=7.3 Hz, 2H), 1.41 (dq, J=14.9, 7.0 Hz, 4H), 1.33-1.09 (m, 55H), 0.88 (td, J=6.9, 2.0 Hz, 10H).

13C NMR (101 MHz, CDCl3) δ 174.21, 172.80, 172.24, 138.79, 138.52, 138.27, 135.34, 128.72, 128.66, 128.64, 128.53, 128.40, 128.26, 128.10, 128.06, 127.89, 127.64, 127.59, 127.51, 103.82, 92.52, 82.12, 79.15, 75.18, 75.09, 74.94, 74.20, 73.75, 73.69, 73.44, 73.24, 72.79, 69.87, 69.81, 67.94, 62.16, 52.74, 36.77, 33.91, 33.86, 31.93, 29.69, 29.64, 29.51, 29.47, 29.36, 29.33, 29.29, 29.23, 29.11, 29.00, 25.59, 24.59, 24.39, 22.70, 14.12.

### Biology

The ability of compounds FP20, FP21, FP22, FP23 and FP24 to selectively activate TLR4 was initially investigated on specific HEK reporter cell lines. HEK-BIue™ hTLR4 and HEK-BIue™ hTLR2 (InvivoGen) are cell lines designed to study the activation of human TLR4 and TLR2 receptors, respectively, by monitoring the activation of transcription factors NF-kB and AP-1. Stimulation with TLR4 ligands (in the case of HEK-Blue hTLR4) or with TLR2 ligands (in the case of HEK-Blue hTLR2) activates NF-kB and AP-1, inducing the production and release of the SEAP reporter gene (phosphatase secreted embryonic alkaline) in the extracellular environment. The analysis of the reporter gene was performed using the QUANTI-Blue™ colorimetric assay (InvivoGen), a substrate of SEAP, which generates a chromogenic product whose absorbance is read at 630 nm. The agonist activity of the molecules was tested by treating HEK-Blue hTLR4 cells for 18 hours with increasing concentrations of the compounds (0.1-1-10-25 μM) and using MPLA (0.1-1-10 μM) and S-LPS (100 ng/mL) as a reference and positive receptor activation control, respectively. The results obtained show that the molecules FP20, FP21, FP22, FP23 and FP24 are able to induce the activation of TLR4 in a dose-dependent manner (FIG. 1a). The molecules were subsequently tested on the HEK-Blue hTLR2 cell line with the aim of excluding the activation of this receptor. In this regard, HEK-Blue hTLR2 cells were treated with the same compounds and at the same concentrations as the tests conducted previously and the compound PAM2CSK4 was used as a positive control for TLR2 activation. As expected, stimulation with PAM2CSK4 induced a strong activation of TLR2, while treatment with compounds FP20, FP21, FP22, FP23 and FP24 did not produce any activation (FIG. 1b).

Following the results obtained from screening tests on HEK cells, the biological activity of compounds FP20, FP21, FP22, FP23 and FP24 was investigated in human and mouse macrophage cell lines. THP-1-X BIue™ cell lines, monocytes differentiated into macrophages following treatment with PMA 100 ng/mL, and RAW-BIue™ were used. Similarly to HEK-Blue cells, THP-1 X-Blue and RAW-Ox stably express the SEAP reporter gene, under the control of transcription factors NF-kB and AP-1. The cells were treated as previously described. The results show that all compounds induce the activation of NF-kB, both on human (FIG. 2a) and murine macrophages (FIG. 2b); the FP23 compound is the only one statistically insignificant on the human macrophage line. Furthermore, on the RAW-blue line the compounds are active at the lowest concentration tested (0.1 μM), while on the THP-1-X-Blue the compounds activate significantly starting from a concentration 100 times higher (10 μM).

To evaluate the cytotoxicity of compounds FP20, FP21, FP22, FP23 and FP24, THP-1-X-Blue cells differentiated into macrophages (FIG. 3) and RAW-Blue (FIG. 4) were treated with increasing concentrations of the test compounds (0.1, 1, 10, 25, 50 μM). The toxicity of the compounds was evaluated by MTT viability assay (3-(4,5-dimethylthiazol-2-yl)-2,5-diphenyltetrazolium bromide). The results obtained show that the compounds are non-toxic on the THP-1-X-Blue cell line, however at the highest concentration tested (50 μM) there is a decrease in cell viability following treatment with FP20 and FP23 (FIG. 3). The results on the mouse macrophage line show an increase in the toxicity of the compounds at a concentration of 50 μM, while the compound FP23 is the only one to show toxicity at a concentration of 25 μM.

FP200 activity was then assessed on human monocytes cell line THP-1-X-Blues, described before. Cells were treated with compounds FP11, FP112, FP20, FP200 and FP21 in increasing concentrations (0.1, 1, 10, 20 μM) and using MPLA (0.1-1-10 μM) and S-LPS (100 ng/mL) as a reference and positive receptor activation control, respectively. The result shows that all compounds induce the activation of NF-kB in a dose dependent manner.

Compound FP207, functionalized with with MPA, was tested in vitro on human THP-1-XBlue cells to evaluate the NF-kB activation. The molecule was tested at the same concentrations used for FP22 and FP23.

Also in this case, the results revealed that compound FP207 is active on TLR4 and induces the production of the transcription factor. Its agonist activity has a concentration-dependent manner as the previous case. Astonishingly, it is more active than FP20 and also comparable to LPS at 25 μM. This is a great result because it will be possible to use less quantity of product to obtain the same inflammatory effect compared to FP20, which will be advantageous in two ways: pharmacologically any possible collateral effect is reduced; while economically this means that less expenses are required to achieve greater results and larger public.

A preliminary test to investigate the toxicity of this functionalized compound was performed, even though without a triplicate. However preliminary, those data suggest that the molecule is non-toxic in the concentration range from 1 to 25 mM.

In vitro and vivo data regarding the compounds disclosed in WO2019/092572 are provided below.

### TLR4 Activation by Synthetic Agonists

The ability of FP molecules to activate human TLR4 was assessed using HEK-Blue hTLR4 cells. These are a HEK293-derived cell line stably transfected with the LPS receptors CD14, TLR4 and MD-2 and a reporter gene, secreted embryonic alkaline phosphatase (SEAP) placed under the control of two TLR4-dependent transcription factors (NF-kB and AP-1). The HEK-Blue hTLR4 cells were treated with increasing concentrations (0.1-25 μM) of FP11, FP112 and FP111 over 18 hours. Stimulation with smooth chemotype of LPS (S-LPS) served as a positive control for the activation of the TLR4-mediated pathway.

Molecules FP11 and FP112 induced the release of the SEAP reporter protein in the medium in a concentration-dependent manner, indicating that both compounds activate NF-kB andAP-1, while FP111 was inactive (FIG. 5A). The three compounds did not inhibit LPS-induced SEAP production, suggesting they lack a TLR4 antagonistic activity (FIG. 5B). A lack of activity on HEK-Blue Null cells, which carry the same SEAP reporter gene but lack the LPS receptors, confirmed that both FP11 and FP112 act via TLR4 (FIG. 5C). To confirm the selectivity on TLR4 over TLR2, the molecules were also tested on the HEK-Blue cells expressing the human Toll-like receptor 2 (hTLR2), resulting in no agonist activity (FIG. 5D). These data agree with the in vitro binding results and suggest that FP11 and FP112 are specific TLR4 agonists that directly interact with the co-receptor MD-2.

### Adjuvant Activity of FP11 and FP112 and In Vivo Toxicity: OVA Immunization Experiments

The ability of FP11 and FP112 to induce immune responses in vivo was compared to MPLA by evaluating antibody production in C₅₇Bl/6 mice immunized with chicken ovalbumin (OVA) as a model antigen. Has been first evaluated the toxicity of FPs in a pilot experiment in which mice were injected subcutaneously with 10 μg of the FP11 and FP112. The results showed that the two test adjuvants had no obvious adverse effect on mice, as assessed by the local response at the injection site and by determining the animal weight and state of alertness over 7 days (FIG. 6A). Next, mice were immunized with the tested adjuvants mixed with ovalbumin (OVA). The induction of antibody was evaluated 21 days post immunization. The results showed that mice immunized with the test adjuvants exhibited marginally higher levels of anti-OVA total IgG after prime immunization compared to OVA-immunized control and significantly lower levels compared to MPLA-OVA immunized animals (FIG. 6B, Prime Immunization). In contrast, after a boost immunization given on day 22 and examined for ova-specific antibody titres 14 days later, the IgG levels in the FP112-immunized mice were higher than those in the FP11-immunized group (FIG. 6B, Booster immunization). These data indicate that in agreement with in vitro and in cell results, FP112 is a more effective adjuvant in vivo than FP11 and has a potency comparable or even greater than MPLA.

### Impurity 1 of Step 6 in FP11 Synthesis, Reference FIG. 7

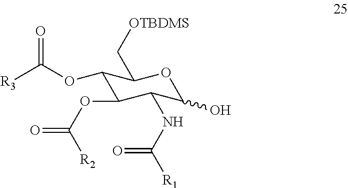

¹H NMR (400 MHz, CDCl₃) δ 6.04 (d, J=9.7 Hz, 1H), 5.63 (dd, J=9.7, 7.3 Hz, 1H), 3.87 (dt, J=7.5, 3.9 Hz, 1H), 3.77-3.70 (m, 3H), 3.68 (dd, J=10.5, 4.3 Hz, 1H), 2.36 (dd, J=14.7, 6.9 Hz, 2H), 2.32-2.26 (m, 3H), 1.67 (dt, J=15.3, 7.7 Hz, 2H), 1.59 (dt, J=20.5, 7.1 Hz, 4H), 1.27 (d, J=16.5 Hz, 62H), 0.91-0.84 (m, 20H), 0.05 (d, J=8.4 Hz, 7H).

Impurity 2 of step 6 in FP11 Synthesis, reference FIG. 8 (¹H NMR) and 9 (¹³C NMR)

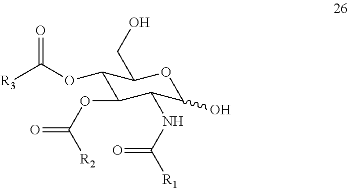

¹H NMR (400 MHz, CDCl₃) δ 6.01 (d, J=9.1 Hz, 1H), 5.40-5.30 (m, 1H), 5.21 (t, J=6.7 Hz, 1H), 5.10-4.99 (m, 2H), 4.22 (td, J=10.7, 3.4 Hz, 1H), 4.04 (ddd, J=10.2, 4.3, 2.0 Hz, 1H), 3.68 (d, J=11.7 Hz, 1H), 3.61-3.53 (m, 1H), 2.32-2.19 (m, 5H), 2.18-2.06 (m, 3H), 1.62-1.46 (m, 8H), 1.34-1.18 (m, 62H), 0.87 (t, J=6.8 Hz, 11H).

¹³C NMR (101 MHz, CDCl₃) δ 174.14, 173.65, 173.12, 91.40, 77.32, 77.01, 76.69, 70.37, 69.67, 68.54, 61.19, 52.49, 36.68, 34.19, 34.13, 31.90, 29.66, 29.63, 29.60, 29.54, 29.52, 29.46, 29.39, 29.34, 29.33, 29.29, 29.27, 29.19, 29.15, 25.60, 24.92, 23.82, 22.66, 14.08.

1. A compound of formula 1 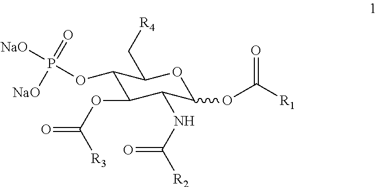
wherein R₁ is a saturated C₅-C₁₅ alkyl chain,
wherein R₂ is a saturated C₅-C₁₅ alkyl chain,
wherein R₃ is a saturated C₅-C₁₅ alkyl chain,
wherein R₄ is any substituent that can be linked by means of a bond between C₆ and a suitable atom and/or any substituent which possesses an oxygen or a nitrogen atom that can bind to C₆.

2. The compound according to claim 1, wherein R₄ chain is an hydroxyl group (OH), a phosphate group (PO₄²⁻), an azide group (N₃), an amine group (NH₂), an acyl group (O(C═O)R) or an alkyl group (OR) or a glycosyl group.

3. The compound according to claim 1, wherein R₁, R₂, R₃, differ from each other.

4. The compound according to claim 1, wherein at least two of R₁, R₂, and R₃, are identical.

5. The compound according to claim 1, wherein at least one of R₁, R₂, and R₃, is free from —OH substituents on position 2.

6. The compound according to claim 1, wherein at least one of R₁, R₂ or R₃, is free from any substituent.

7. The compound according to claim 1, wherein
R₁=R₂=R₃=C₁₁H₂₃ and R₄═OH, or
R₁=R₃=C₁₃H₂₇; R₂=C₁₁H₂₃ and R₄═OH, or
R₁=R₂=R₃=C₉H₁₉ and R₄═OH, or
R₁=R₂=R₃=C₁₃H₂₇ and R₄═OH, or
R₁=R₃=C₉H₁₉; R₂=C₁H₂₃ and R₄═OH, or
R₁=R₂=R₃=C₁₁H₂₃ and R₄=PO₄²⁻, or
R₁=R₂=R₃=C₉H₁₉ and R₄=PO₄²⁻, or
R₁=R₂=R₃=C₁₃H₂₇ and R₄=PO₄²⁻, or
R₁=R₂=R₃=C₁₁H₂₃ and R₄=OC₃H₇, or
R₁=R₂=R₃=C₁₁H₂₃ and R₄=O(C═O)C₆H₈(OH)₃, or
R₁=R₂=R₃=C₁₁H₂₃ and R₄=NH₂
R₁=R₂=R₃=C₁₁H₂₃ and R₄=O(C═O)CCH₃(CH₂OH)₂
R₁=R₂=R₃=C₁₁H₂₃ and R₄=OCH(CHOH)₃CH(CH₃)O
R₁=R₂=R₃=C₁₁H₂₃ and R₄=OCH(CHOH)₃CH(CH₂OH)O
R₁=R₂=R₃=C₁₁H₂₃ and R₄=OCH(CHOH)₃(CH₂)O.

8. The compound of claim 1 wherein said compound is an α anomer of the compound of formula 1.

9. The compound of claim 1 wherein said compound is an β anomer of the compound of formula 1.

10. A method of treating a disease that requires or benefits from immunostimulation by activating the TLR4 receptor comprising administering to a subject a therapeutically effective amount of the compound according to claim 1.

11. The method according to claim 10 wherein said diseases are cancer, allergies, infectious diseases, cardiovascular diseases, obesity-dependent metabolic diseases, neuronal degeneration, apoptosis, autoimmune disorders, viral infections, bacterial infections, autoimmune diseases.

12. A vaccine adjuvant consisting of the compound as defined in claim 14.

13. A vaccine composition comprising the compound as defined in claim 1, at least one pharmaceutically acceptable carrier and at least one pharmaceutically acceptable immunogenic antigen.

14. The vaccine composition according to claim 13, wherein said compound is the sole adjuvant present in said composition.

15. A pharmaceutical composition comprising the compound as defined in claim 1 and at least one pharmaceutically acceptable excipient and/or carrier.

16. The pharmaceutical composition according to claim 15, further comprising at least one additional active principle.

17. The pharmaceutical composition according to claim 15, in a form for oral, parenteral, nasal, aerosol, sublingual, rectal, vaginal, topical or systemic administration.

18. The pharmaceutical composition according to claim 15 in the form of suspension, emulsion, ointment, cream, spray, granulate, powder, solution, capsule, pill, tablet, lyophilized product, lozenge, aerosol, nebulization, or injection.

19. A method of treatment of diseases that require or benefit from an immunostimulation by activating the TLR4 receptor, comprising administering the pharmaceutical composition of claim 15 to a subject in need thereof.

20. The method according to claim 19, wherein said diseases are cancer, allergies, infectious diseases, cardiovascular diseases, obesity-dependent metabolic diseases, neuronal degeneration, apoptosis, autoimmune disorders, viral infections, bacterial infections, autoimmune diseases.

21. An intermediate of formula 1i 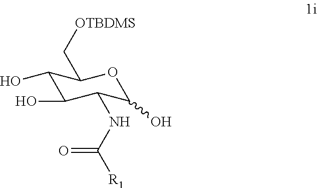
wherein R₁ is a saturated C₅-C₁₅ alkyl chain.

22. A method for the preparation of an intermediate of formula 1i 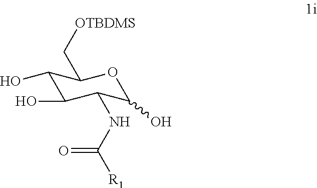
comprising the following steps
1) Selective acylation of the amino group in the C₂ position of glucosamine hydrochloride by reaction with acyl chloride in the presence of sodium bicarbonate.
2) Protection by selective silylation of hydroxyl in position C₆ by reaction with tert-butyldimethylsilyl chloride (TBDMSCI) in the presence of imidazole.

23. A method for the preparation of a compound of formula 1, 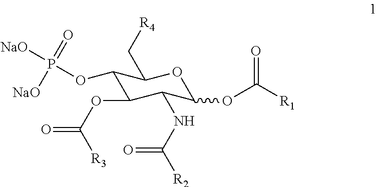
wherein R₁ is a saturated C₅-C₁₅ alkyl chain,
wherein R₂ is a saturated C₅-C₁₅ alkyl chain,
wherein R₃ is a saturated C₅-C₁₅ alkyl chain,
wherein R₄ is any substituent that can be linked by means of a bond between C₆ and a suitable atom and/or any substituent which possesses an oxygen or a nitrogen atom that can bind to C₆, comprising the following steps:
1) Selective acylation of the amino group in the C₂ position of glucosamine hydrochloride by reaction with acyl chloride in the presence of sodium bicarbonate;
2) Protection by selective silylation of hydroxyl in position C₆ by reaction with tert-butyldimethylsilyl chloride (TBDMSCI) in the presence of imidazole, thereby obtaining the intermediate as defined in claim 22;
3) Selective acylation of hydroxyls in positions C₁ and C₃ by reaction with acyl chloride in the presence of triethylamine and N, N-dimethyl aminopyridine (DMAP);
4) Phosphorylation of hydroxyl in the C₄ position by reaction with dibenzyl N, N-diisopropylphospharamidite in the presence of triflate imidazolium, followed by oxidation of phosphite to phosphate via metachloroperbenzoic acid;
5) Deprotection of hydroxyl from silane in position C₆ through the presence of sulfuric acid in catalytic quantities; and
6) Deprotection of phosphate from benzyls in position C₄ and optionally deprotection of benzyls on any substituent in position C₆ through hydrogenation catalyzed by Palladium on Carbon (Pd/C).

24. The method of claim 23, further comprising a step 5i) after step 5) and before step 6):
5i) Phosphorylation of hydroxyl in position C₆ by reaction with dibenzyl N, N-diisopropylphospharamidite in the presence of triflate imidazolium, followed by oxidation of phosphite to phosphate via metachloroperbenzoic acid wherein said deprotection step 6) is carried out in position C₄ and C₆, and
wherein the resulting R₄ is a phosphate group (PO₄²⁻).

25. The method of claim 23 wherein the compound of formula 1 is one of
R₁=R₂=R₃=C₁₁H₂₃ and R₄=PO₄²⁻, or
R₁=R₂=R₃=C₉H₁₉ and R₄=PO₄²⁻, or
R₁=R₂=R₃=C₁₃H₂₇ and R₄=PO₄²⁻.

26. The method of claim 23, further comprising a step 5ii) after step 5) and before step 6):
5ii) Acylation of hydroxy in C₆ position by reaction with carboxylic acid in the presence of a suitable condensing agent and catalyst, or with acyl chloride in the presence of a suitable catalyst, wherein said deprotection step 6) is carried out in position C₄ and
wherein the resulting R₄ is an acyl group.

27. The method of claim 26 wherein in said acylation step 5ii) said suitable condensing agent and catalyst are 1-ethyl-3-(3-dimethylaminopropyl) carbodiimide (EDC) and N, N-dimethyl aminopyridine (DMAP).

28. The method of claim 23, further comprising a step 5iii) after step 5) and before step 6):
5iii) Glycosylation of hydroxyl in C₆ position by reaction of a glycosyl chloride donor or of a glychoside tioethyl (Set) donor in the presence of a suitable activator, catalyst and a molecular sieve wherein the resulting R₄ is a glycosyl group.

29. The method of claim 23, further comprising a step 5iv) after step 5) and before step 6):
5iv) Alkylation of hydroxyl in C₆ by reaction of a stabilized alkyl chloride in the presence of a suitable activator, catalyst and a molecular sieve
wherein the resulting R₄ is n alkyl group.

30. The method of claim 28, wherein in said glycosylation step 5iii) and alkylation step 5iv) said suitable activator is silver (I) oxide or NIS (N-iodosuccinimide), said suitable catalyst is trifilic acid or HOFox (3,3-difluoroxindole), and said suitable molecular sieve is a water scavenger.

31. The method of claim 23, further comprising a step 5v) and a step 5vi) after step 5) and before step 6):
5v) Tosylation of position C₆ by reaction of tosyl chloride in presence of triethylamine as base and of a suitable catalyst
5vi) Azide instertion in position C₆ by reaction with sodium azide in the presence of tetrabutylammonium iodide.

32. The method of claim 31, wherein in said tosylation step 5v) said suitable catalyst is N, N-dimethyl aminopyridine (DMAP).

33. The method of claim 23, further comprising a step 5vii) and a step 5viii) after step 5) and before step 6):
5vii) Glycosylation of hydroxyl in C₆ position by reaction of a glycosyl chloride donor bearing a picoloyl group in the presence of Bi(OTf)₃ as sole activator.
wherein the resulting R₄ is a glycosyl group.
5viii) Picoloyl group removal by reaction with Cu(OAc)₂.

34. The method of claim 24 wherein said acylation step 3) is carried out at a temperature ranging from −78° C. of 0° C. and an amount of catalyst ranging from 0.05 to 0.2 equivalents thereby obtaining a β-anomer of said compound of formula 1, preferably a temperature of −20° C. and an amount of catalyst of 0.1 equivalents.

35. The method of claim 24, wherein said acylation step 3) is carried out at a temperature ranging from 20° C. to 50° C. and an amount of catalyst ranging from 2 to 2.5 equivalents thereby obtaining an α-anomer of said compound of formula 1, preferably a temperature of 30° C. and an amount of catalyst of 2.02 equivalents.

36. Use of an intermediate compound as defined in claim 21 for the synthesis of compounds of formula 1, 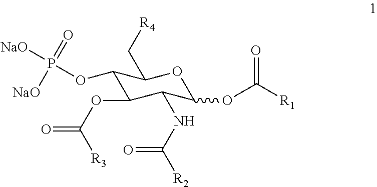
wherein R₁ is a saturated C₅-C₁₅ alkyl chain,
wherein R₂ is a saturated C₅-C₁₅ alkyl chain,
wherein R₃ is a saturated C₅-C₁₅ alkyl chain,
wherein R₄ is any substituent that can be linked by means of a bond between C₆ and a suitable atom and/or any substituent which possesses an oxygen or a nitrogen atom that can bind to C₆.

37. A method for the preparation of a compound of formula X 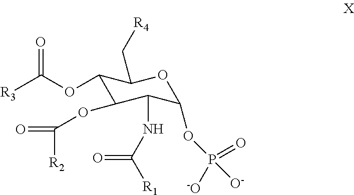
wherein R₁ is a saturated C₅-C₁₅ alkyl chain,
wherein R₂ is a saturated C₅-C₁₅ alkyl chain,
wherein R₃ is a saturated C₅-C₁₅ alkyl chain,
wherein R₄ is OH and wherein each of R₁, R₂ and R₃ is free from —OH substituents in position C₂ comprising the following steps:
1) Selective acylation of the amino group in the C₂ position of glucosamine hydrochloride by reaction with acyl chloride in the presence of sodium bicarbonate.
2) Protection by selective silylation of hydroxyl in position C₆ by reaction with tert-butyldimethylsilyl chloride (TBDMSCI) in the presence of imidazole obtaining the intermediate of formula 1i as defined in claim 21.
3) Complete acylation of hydroxyls in positions C₁, C₃ and C₄ by reaction with acyl chloride in the presence of triethylamine and N, N-dimethyl aminopyridine (DMAP).
4) selective diacylation of position C₁ by reaction with ethylendiamine in presence of acetic acid
5) Phosphorylation of hydroxyl in the C₁ position by reaction with dibenzyl N, N-diisopropylphospharamidite in the presence of triflate imidazolium, followed by oxidation of phosphite to phosphate via metachloroperbenzoic acid.
6) Deprotection of hydroxyl from silane in position C₆ through the presence of sulfuric acid in catalytic quantities.
7) Deprotection of phosphate from benzyls in position C₄ and optionally deprotection of benzyls on any substituent in position C₆ through hydrogenation catalyzed by Palladium on Carbon (Pd/C).

38. Use of an intermediate compound as defined in claim 21 for the synthesis of compounds of formula X, 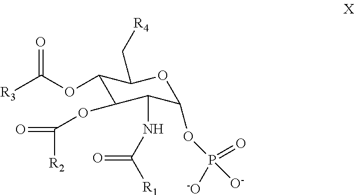
wherein R₁ is a saturated C₅-C₁₅ alkyl chain,
wherein R₂ is a saturated C₅-C₁₅ alkyl chain,
wherein R₃ is a saturated C₅-C₁₅ alkyl chain,
wherein R₄ is OH and wherein each of R₁, R₂ and R₃ is free from —OH substituents in position C₂.
